(19) United States
(12) Patent Application Publication
(10) Pub. No.: US 2025/0302382 A1
(43) Pub. Date: Oct. 2, 2025

(54) Hemodynamic Sensor Systems for Predicting and Diagnosing Hypotension and Characterizing Interventions Thereof

(71) Applicant: Becton, Dickinson and Company, Franklin Lakes, NJ (US)

(72) Inventors: Xianfu Liu, Irvine, CA (US); Zhongping Jian, Irvine, CA (US); Feras Al Hatib, Huntington Beach, CA (US); Sai Prasad Buddi, Costa Mesa, CA (US)

(21) Appl. No.: 19/097,105

(22) Filed: Apr. 1, 2025

Related U.S. Application Data
(60) Provisional application No. 63/572,831, filed on Apr. 1, 2024.

(51) Int. Cl. A61B 5/00; A61B 5/022; A61M 5/172; G16H 10/60; G16H 20/17; G16H 50/30

(52) U.S. Cl. CPC A61B 5/4848; A61B 5/02241; A61B 5/7221; A61B 5/7225; A61B 5/725; A61B 5/7278; A61B 5/7435; A61B 5/746; A61B 5/7475; A61M 5/1723; G16H 10/60; G16H 20/17; G16H 50/30

(57) ABSTRACT

A hemodynamic sensor system can determine a likelihood of effectiveness of the intervention for hypotension of the patient. The system can receive, from a hemodynamic sensor, a test analog hemodynamic sensor signal from the patient and convert, using an analog-to-digital converter, the test analog hemodynamic sensor signal to a test arterial pressure signal waveform. The system can extract from the test arterial pressure signal waveform a plurality of test heart health parameters from a set of available heart health parameters and generate, using a plurality of reference arterial pressure signal waveforms from a plurality of patients, one or more filtered sets of reference heart health parameters. The system can consolidate the filtered sets of reference heart health parameters into corresponding reference feature pools and determine normalized test maximum features from the plurality of test heart health parameters. The system can determine the likelihood of effectiveness of the intervention.

### CROSS REFERENCE TO RELATED APPLICATIONS

This application claims the benefit of and priority to U.S. Provisional Patent Application No. 63/572,831, entitled “HEMODYNAMIC SENSOR SYSTEMS FOR PREDICTING AND DIAGNOSING HYPOTENSION AND CHARACTERIZING INTERVENTIONS THEREOF”, and filed Apr. 1, 2024, the disclosure of which is hereby incorporated by reference in its entirety for all purposes FIELD

The present disclosure relates generally to hemodynamic monitoring, including to determining and predicting and diagnosing hypotension in a patient (e.g., human or veterinary subject) using monitored hemodynamic data.

### BACKGROUND

Monitoring hemodynamic variables of a patient allows for improved patient care. The hemodynamic variables can include heart health parameters, such as cardiac output. Monitoring such heart health parameters can allow a system to make predictions and diagnoses of hypotension and characterize the effectiveness of medical interventions for hypotension in the patients. Systems and methods described herein provide potentially life-saving solutions in the space.

### SUMMARY

In some aspects, systems and methods are disclosed that related to hemodynamic sensor systems for determining a likelihood that a patient with experience hypotension within a threshold period time. Additionally or alternatively, the systems and methods may relate to hemodynamic sensor systems that can estimate a time to event (TTE) of hypotension in a patient.

In some aspects, the techniques described herein relate to a hemodynamic sensor system configured to determine a likelihood of effectiveness of an intervention for hypotension, the system including: a hemodynamic sensor that produces analog hemodynamic sensor signals representative of arterial pressure signal waveforms of patients; an analog-to-digital converter that converts the analog hemodynamic sensor signals to arterial pressure signal waveforms; a graphical user interface configured to display an alert indicating the determined likelihood of effectiveness of the intervention; a non-transitory memory having executable instructions and a deep learning model stored thereon; and an electronic hardware processor in communication with the non-transitory memory and configured to execute the instructions to cause the system to at least: receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters, wherein the set of available heart health parameters includes: a mean arterial pressure (MAP); a stroke volume index (SVI); a hypotension prediction index (HPI); a systemic vascular resistance (SVR); a heart rate (HR); a cardiac output (CO) a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV); obtain a plurality of reference arterial pressure signal waveforms from a plurality of patients; extract from the plurality of reference arterial pressure signal waveforms a plurality of reference sets of heart health parameters, each of the reference sets of heart health parameters including corresponding heart health parameters from the set of available heart health parameters; apply a filter to the reference sets of heart health parameters to generate a feature map associated with the filter; consolidate the filtered sets of reference heart health parameters into corresponding reference feature pools; select a reference maximum feature from each of the reference feature pools; reduce overfitting of the reference feature pools by removing one or more of the reference feature pools from remaining reference feature pools; normalize the selected reference maximum features from the reference feature pools; generate, based on the plurality of heart health parameters, one or more feature pools; determine normalized test maximum features from the plurality of test heart health parameters; determine, based on a comparison between the normalized selected reference maximum features and the normalized test maximum features, that an intervention for hypotension has been implemented and the likelihood of effectiveness of the intervention for hypotension of the patient; and generate, based on the determined likelihood of effectiveness of the intervention for hypotension of the patient, data for displaying, via the graphical user interface, the alert indicating the likelihood of effectiveness of the intervention for hypotension.

In some aspects, the techniques described herein relate to a hemodynamic sensor system configured to determine a likelihood of effectiveness of an intervention for hypotension, the system including: a hemodynamic sensor that produces analog hemodynamic sensor signals representative of arterial pressure signal waveforms of patients; an analog-to-digital converter that converts the analog hemodynamic sensor signals to arterial pressure signal waveforms; a non-transitory memory having executable instructions and a deep learning model stored thereon; and an electronic hardware processor in communication with the non-transitory memory and configured to execute the instructions to cause the system to at least: receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters, wherein the set of available heart health parameters includes: a mean arterial pressure (MAP); a stroke volume index (SVI); a hypotension prediction index (HPI); a systemic vascular resistance (SVR); a heart rate (HR); a cardiac output (CO) a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV); generate, using a plurality of reference arterial pressure signal waveforms from a plurality of patients, one or more filtered sets of reference heart health parameters associated with the plurality of reference arterial pressure signal waveforms; consolidate the filtered sets of reference heart health parameters into corresponding reference feature pools; determine, based on the reference feature pools, normalized test maximum features from the plurality of test heart health parameters; determine, based on a comparison between the normalized selected reference maximum features and the normalized test maximum features, that an intervention for hypotension has been implemented and the likelihood of effectiveness of the intervention for hypotension of the patient; and generate, based on the determined likelihood of effectiveness of the intervention for hypotension of the patient, data for displaying, via the graphical user interface, the alert indicating the likelihood of effectiveness of the intervention for hypotension.

In some aspects, the techniques described herein relate to a hemodynamic sensor system, wherein generating the one or more filtered sets of reference heart health parameters includes: obtaining the plurality of reference arterial pressure signal waveforms from the plurality of patients; extracting from the plurality of reference arterial pressure signal waveforms a plurality of reference sets of heart health parameters, each of the reference sets of heart health parameters including corresponding heart health parameters from the set of available heart health parameters; and applying a filter to the reference sets of heart health parameters to generate a feature map associated with the filter.

In some aspects, the techniques described herein relate to a hemodynamic sensor system, wherein determining the normalized test maximum features from the plurality of test heart health parameters includes: selecting a reference maximum feature from each of the reference feature pools; reducing overfitting of the reference feature pools by removing one or more of the reference feature pools from remaining reference feature pools; normalizing the selected reference maximum features from the reference feature pools; and generating, based on the plurality of heart health parameters, one or more feature pools.

In some aspects, the techniques described herein relate to a hemodynamic sensor system, further including a graphical user interface configured to display the alert indicating the determined likelihood of effectiveness of the intervention.

In some aspects, the techniques described herein relate to a hemodynamic sensor system, wherein the electronic hardware processor is further configured to execute the instructions to cause the system to at least: generate, based on the determined likelihood of effectiveness of the intervention, data for displaying at least the alert indicating the likelihood of effectiveness of the intervention for hypotension.

In some aspects, the techniques described herein relate to a hemodynamic sensor system, wherein the alert includes at least one of a symbol, a numerical value, a visual design, a repeated indicator, or a highlight.

In some aspects, the techniques described herein relate to a hemodynamic sensor system, wherein the electronic hardware processor is further configured to execute the instructions to cause the system to at least: determine, based on the comparison between the normalized selected reference maximum features and the normalized test maximum features, that the patient will experience hypotension within a specified time with at least a target threshold confidence level.

In some aspects, the techniques described herein relate to a hemodynamic sensor system, wherein the electronic hardware processor is further configured to execute the instructions to cause the system to at least: generate, based on the determination that that the patient will experience hypotension within the specified time with at least the target threshold confidence level, data for displaying, via the graphical user interface, the alert indicating the expected time to event of hypotension within the patient.

In some aspects, the techniques described herein relate to a hemodynamic sensor system configured to determine that a patient will experience hypotension within a specified time with at least a target threshold confidence level, the system including: a hemodynamic sensor that produces analog hemodynamic sensor signals representative of arterial pressure signal waveforms of patients; an analog-to-digital converter that converts the analog hemodynamic sensor signals to arterial pressure signal waveforms; a non-transitory memory having executable instructions and a deep learning model stored thereon; and an electronic hardware processor in communication with the non-transitory memory and configured to execute the instructions to cause the system to at least: receive the specified time and receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters, wherein the set of available heart health parameters includes: a mean arterial pressure (MAP); a stroke volume index (SVI); a hypotension prediction index (HPI); a systemic vascular resistance (SVR); a heart rate (HR); a cardiac output (CO) a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV); generate, using a plurality of reference arterial pressure signal waveforms from a plurality of patients, one or more filtered sets of reference heart health parameters associated with the plurality of reference arterial pressure signal waveforms; consolidate the filtered sets of reference heart health parameters into corresponding reference feature pools; determine, based on the reference feature pools, normalized test maximum features from the plurality of test heart health parameters; determine, based on a comparison between the normalized selected reference maximum features and the normalized test maximum features, that the patient will experience hypotension within the specified time with at least the target threshold confidence level; and generate, based on the determination that that the patient will experience hypotension within the specified time with at least the target threshold confidence level, data for displaying, via the graphical user interface, the alert indicating the expected time to event of hypotension within the patient.

In some aspects, the techniques described herein relate to a hemodynamic sensor system, wherein generating the one or more filtered sets of reference heart health parameters includes: obtaining the plurality of reference arterial pressure signal waveforms from the plurality of patients; extracting from the plurality of reference arterial pressure signal waveforms a plurality of reference sets of heart health parameters, each of the reference sets of heart health parameters including corresponding heart health parameters from the set of available heart health parameters; and applying a filter to the reference sets of heart health parameters to generate a feature map associated with the filter.

In some aspects, the techniques described herein relate to a hemodynamic sensor system, wherein determining the normalized test maximum features from the plurality of test heart health parameters includes: selecting a reference maximum feature from each of the reference feature pools; reducing overfitting of the reference feature pools by removing one or more of the reference feature pools from remaining reference feature pools; normalizing the selected reference maximum features from the reference feature pools; and generating, based on the plurality of heart health parameters, one or more feature pools.

In some aspects, the techniques described herein relate to a hemodynamic sensor system, further including a graphical user interface configured to display the alert indicating an expected time to event of hypotension within the patient.

In some aspects, the techniques described herein relate to a hemodynamic sensor system configured to determine that a patient will experience hypotension within a specified time with at least a target threshold confidence level, the system including: a hemodynamic sensor that produces analog hemodynamic sensor signals representative of arterial pressure signal waveforms of patients; an analog-to-digital converter that converts the analog hemodynamic sensor signals to arterial pressure signal waveforms; a graphical user interface configured to display an alert indicating an expected time to event of hypotension within the patient; a non-transitory memory having executable instructions and a deep learning model stored thereon; and an electronic hardware processor in communication with the non-transitory memory and configured to execute the instructions to cause the system to at least: receive the specified time and receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters, wherein the set of available heart health parameters includes: a mean arterial pressure (MAP); a stroke volume index (SVI); a hypotension prediction index (HPI); a systemic vascular resistance (SVR); a heart rate (HR); a cardiac output (CO) a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV); obtain a plurality of reference arterial pressure signal waveforms from a plurality of patients; extract from the plurality of reference arterial pressure signal waveforms a plurality of reference sets of heart health parameters, each of the reference sets of heart health parameters including corresponding heart health parameters from the set of available heart health parameters; apply a filter to the reference sets of heart health parameters to generate a feature map associated with the filter; consolidate the filtered sets of reference heart health parameters into corresponding reference feature pools; select a reference maximum feature from each of the reference feature pools; reduce overfitting of the reference feature pools by removing one or more of the reference feature pools from remaining reference feature pools; normalize the selected reference maximum features from the reference feature pools; generate, based on the plurality of heart health parameters, one or more feature pools; determine normalized test maximum features from the plurality of test heart health parameters; determine, based on a comparison between the normalized selected reference maximum features and the normalized test maximum features, that the patient will experience hypotension within the specified time with at least the target threshold confidence level; and generate, based on the determination that that the patient will experience hypotension within the specified time with at least the target threshold confidence level, data for displaying, via the graphical user interface, the alert indicating the expected time to event of hypotension within the patient.

In some aspects, the techniques described herein relate to a hemodynamic sensor system configured to determine a degree of certainty that a patient will experience hypotension within a specified time with at least a target threshold confidence level and determine a likelihood of effectiveness of an intervention for the hypotension, the system including: a hemodynamic sensor that produces analog hemodynamic sensor signals representative of arterial pressure signal waveforms of patients; an analog-to-digital converter that converts the analog hemodynamic sensor signals to arterial pressure signal waveforms; a non-transitory memory having executable instructions and a deep learning model stored thereon; and an electronic hardware processor in communication with the non-transitory memory and configured to execute the instructions to cause the system to at least: receive the specified time; receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters; generate, using a plurality of reference arterial pressure signal waveforms from a plurality of patients, one or more filtered sets of reference heart health parameters associated with the plurality of reference arterial pressure signal waveforms; consolidate the filtered sets of reference heart health parameters into corresponding reference feature pools; determine, based on the reference feature pools, normalized test maximum features from the plurality of test heart health parameters; determine, based on a comparison between the normalized selected reference maximum features and the normalized test maximum features, a degree of certainty that the patient will experience hypotension within the specified time with at least the target threshold confidence level; determine, based on the normalized test maximum features, that an intervention for hypotension has been implemented and the likelihood of effectiveness of the intervention for hypotension of the patient; determine a hypotension prediction index (HPI) based on the determination that the patient will experience hypotension within the specified time with at least the target threshold confidence level and based on the likelihood of effectiveness of the intervention for hypotension of the patient; determine that the HPI exceeds a predetermined threshold; and generate, based on the determination that the HPI exceeds the predetermined threshold, the alert for display via a graphical user interface.

In some aspects, the techniques described herein relate to a hemodynamic sensor system, further including the graphical user interface configured to display the alert indicating at least one of an expected time to event of hypotension within the patient or an indication of effectiveness of an intervention.

In some aspects, the techniques described herein relate to a hemodynamic sensor system, wherein the alert is displayed via the graphical user interface.

In some aspects, the techniques described herein relate to a hemodynamic sensor system, wherein the set of available heart health parameters includes: a mean arterial pressure (MAP); a stroke volume index (SVI); a hypotension prediction index (HPI); a systemic vascular resistance (SVR); a heart rate (HR); a cardiac output (CO) a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV).

In some aspects, the techniques described herein relate to a hemodynamic sensor system, wherein generating the one or more filtered sets of reference heart health parameters includes: obtaining the plurality of reference arterial pressure signal waveforms from the plurality of patients; extracting from the plurality of reference arterial pressure signal waveforms a plurality of reference sets of heart health parameters, each of the reference sets of heart health parameters including corresponding heart health parameters from the set of available heart health parameters; and applying a filter to the reference sets of heart health parameters to generate a feature map associated with the filter.

In some aspects, the techniques described herein relate to a hemodynamic sensor system, wherein determining the normalized test maximum features from the plurality of test heart health parameters includes: selecting a reference maximum feature from each of the reference feature pools; reducing overfitting of the reference feature pools by removing one or more of the reference feature pools from remaining reference feature pools; normalizing the selected reference maximum features from the reference feature pools; and generating, based on the plurality of heart health parameters, one or more feature pools.

### BRIEF DESCRIPTION OF THE DRAWINGS

FIG. 1 is a block diagram of a hemodynamic sensing system that can determine a likelihood that a patient will experience hypotension within a certain period of time based on hemodynamic signal data, according to some embodiments.

FIG. 2 is a graph illustrating an example arterial pressure signal waveform.

FIG. 3 is a perspective view of an example hemodynamic sensor that can be coupled (e.g., attached) to the patient for sensing hemodynamic data representative of arterial pressure of the patient, according to some embodiments.

FIG. 4 is a perspective view of an example hemodynamic sensor for sensing hemodynamic data representative of arterial pressure of the patient, according to some embodiments.

FIG. 5 shows an example architecture of a deep learning model 500 that may be used, for example, by the machine learning model 124.

FIG. 6A shows a graph of example results of calculating a goodness of clustering across a potential number of clusters in latent space, ranging from 2 to 14 clusters.

FIG. 6B shows a graph of an example evaluation of a goodness of clustering of a set of locations in original space using a clustering evaluation metric.

FIGS. 7A-7C show relationships between a determined HPI and the TTE over time, as determined by various embodiments of the systems and deep learning models described herein.

FIG. 8A shows a flowchart of an example method 800 for determining a time to event (TTE) and/or a likelihood of effectiveness of an intervention of hypotension.

FIG. 8B shows a flowchart of an example method 850 for generating one or more feature pools.

FIG. 9 is a block diagram that illustrates a computer system upon which various implementations may be implemented.

### DETAILED DESCRIPTION

### Overview

Hypotension, or low blood pressure, is a physiological condition characterized by a blood pressure reading below an acceptable threshold level. An acceptable threshold level of blood pressure may be generally considered to be at least 90/60 mm Hg for most people (e.g., 90 mm Hg for a systolic pressure threshold, and 60 mm Hg for a diastolic pressure threshold level), below which may be considered hypotension for most people. The definition of hypotension can vary based on the individual circumstances, so some people may naturally have lower blood pressure without experiencing any hypotensive symptoms. Like its counterpart of hypertension, hypotension can also pose health risks and warrant serious medical attention. Blood pressure is a vital component of cardiovascular health, and deviations from an acceptable range can impact organ perfusion and overall well-being.

Traditionally, the diagnosis of hypotension involves measuring blood pressure using a sphygmomanometer. A systolic pressure below a systolic pressure threshold level (e.g., about 90 mm Hg) and/or a diastolic pressure below a diastolic pressure threshold level (e.g., about 60 mm Hg) may be considered indicative of hypotension. However, this conventional approach might overlook underlying complexities that contribute to the condition. Identifying the effectiveness of a treatment of hypotension in a patient can be an important step in properly treating the particular needs of a hypotensive or potentially hypotensive patient. For example, a patient may suffer one or more endotypes of hypotension, each representing a distinct subtype or mechanism that leads to, or results in, hypotension in individual patients. An endotype of hypotension generally refers to a specific type of low blood pressure characterized by distinct physiological or molecular features. A treatment of one endotype may be unsuitable or unsuccessful in treating a different endotype. A treatment may be ineffective for other reasons.

The systems described herein can predict and/or diagnose hypotension in a patient (e.g., human or veterinary subject) using monitored hemodynamic data. The system can determine a degree of certainty that a patient will experience hypotension within a particular amount of time with a particular confidence level. Additionally or alternatively, the system can determine whether an intervention for hypotension has been implemented and/or a likelihood of effectiveness of the intervention. These factors may be used to develop a hypotension prediction index (HPI). The HPI may take into account other variables or factors described herein. If the system determines that the HPI exceeds a predetermined threshold, the system can generate an alert to get the attention of a healthcare worker to modify an intervention type or amount, notify relevant parties, and/or take additional action (e.g., determining a therapy protocol for the patient and/or generating a command to cause an infusion pump to deliver therapy to the patient).

Several embodiments of the invention are particularly advantageous because they include one, several or all of the following benefits: (i) reduce or prevent mistakes in diagnosing and/or treating hypotension, (ii) allow for estimates of effectiveness of an intervention of hypotension in real-time, including during emergency situations, (iii) use a combination of convoluted neural network layers and transformer algorithms to overcome limitations in the function of computers in identifying the effectiveness of hypotension interventions and a likelihood of entering hypotension, and/or (iv) generate real-time output to local and/or remote computing devices based on updated data in real-time.

A hemodynamic sensing or monitoring system can be used to determine whether a patient is likely to experience hypotension within a particular amount of time and/or determine an effectiveness of an intervention for hypotension in real time. Such systems may be more accurate and/or rapid than human analysis. Properly diagnosing (or, in some cases, predicting) when or if hypotension will occur can result in better (e.g., more effective or more rapid) treatment. For example, vasodilation may be characterized by an abnormal widening or dilation of blood vessels. Accordingly, using vasopressors to constrict blood vessels may be appropriate. By contrast, hypovolemia can result from a significant loss of fluid (e.g., blood) from the body. Treatment of hypovolemia may include providing the patient with intravenous fluid, such as saline or colloids. Using an incorrect treatment type for a different endotype of hypotension than is expected may result in no effect on the patient's health or possibly may exacerbate or worsen an already bad health condition.

The hemodynamic sensing system may obtain an analog arterial pressure signal. This may be an analog hemodynamic sensor signal (e.g., analog hemodynamic signal) that can be converted to a different form (e.g., digital form) of signal, such as an arterial pressure signal waveform. The hemodynamic sensing system can use machine learning to extract sets of parameters, such as heart health parameters, from the arterial pressure of the patient. As described herein, “heart health parameters” can have its plain and ordinary meaning and may generally refer to health parameters associated with cardiovascular health (e.g., vascular health, blood health, etc.) and need not be specific to the heart. The sets of input features can be used by the hemodynamic sensing system to determine the effectiveness of a hypotension treatment and/or an estimated time to event (e.g., of hypotension) while the patient is visiting an office of a primary care physician, while in an emergency care setting, and/or in any other patient care environment. In some embodiments, the hemodynamic sensing system can even be made available “over the counter” for use at home by the patient.

Depending on the HPI generated by the hemodynamic sensing system, the hemodynamic sensing system can generate a signal or an alarm to medical workers and/or the patient to alert the medical workers and/or the patient that the patient requires attention (e.g., immediate emergency attention). The alert may include an indication of a modification of hypotension treatment.

### Hemodynamic Sensing System

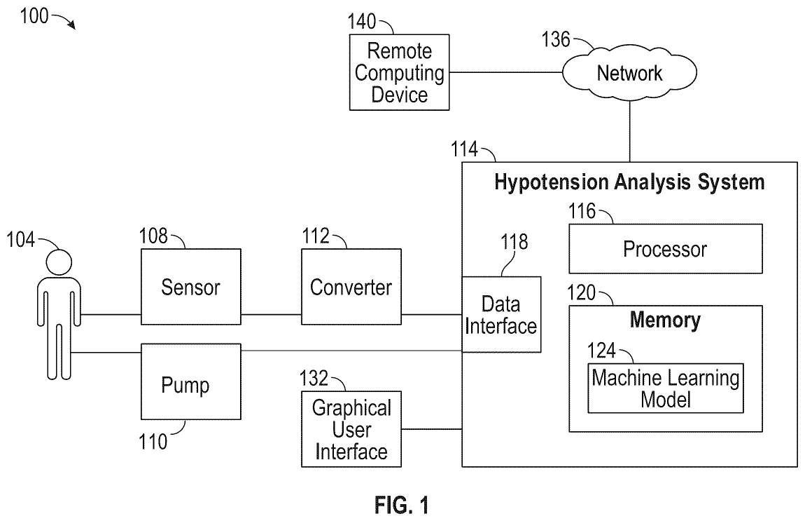

FIG. 1 is a block diagram of a hemodynamic sensing system 100 that can determine a degree of certainty that a patient will experience hypotension within a specified time with at least a target threshold confidence level and/or determine a likelihood of effectiveness of an intervention for the hypotension, according to some embodiments. Additionally or alternatively, the hemodynamic sensing system 100 can generate a hemodynamic prediction index (HPI) to determine whether intervention in a hypotensive or potentially hypotensive patient is required. If the system determines that some intervention is required, the system may determine a therapy protocol for the patient and/or generate a command to cause an infusion pump to deliver therapy to the patient). As illustrated in FIG. 1, the hemodynamic sensing system 100 includes a hemodynamic sensor 108 coupled to a patient 104, a signal converter 112, a hypotension analysis system 114, and/or a graphical user interface 132. In some embodiments, the hemodynamic sensing system 100 includes a remote computing device 140 connected via a network 136. The hypotension analysis system 114 can include one or more processors 116, a hemodynamic data interface 118, and/or a memory 120. The memory 120 can include instructions (e.g., software instructions) stored thereon for implementing one or more steps described herein. Additionally or alternatively, the memory 120 can include a machine learning model 124 and/or other artificial intelligence components. The machine learning model 124 may include a deep learning model. For example, the machine learning model 124 can include a convoluted neural network, a long short-term memory network, a transformer, and/or other elements described herein. The hemodynamic sensing system 100 can be implemented within a patient care environment, such as an intensive care unit (ICU), an operating room (OR), and/or other patient care environment.

For example, in some embodiments, the hemodynamic sensing system 100 includes a hemodynamic sensor 108, a pump 110, a signal converter 112, a memory 120, and one or more processors 116. The one or more processors 116 are configured to execute instructions stored on the memory 120 to receive, from the hemodynamic sensor 108, an analog hemodynamic sensor signal from a patient. The one or more processors 116 can cause the hemodynamic sensing system 100 to convert, using the signal converter 112, the analog hemodynamic sensor signal to the arterial pressure signal waveform and determine, based on the arterial pressure signal waveform, an estimated time of event (e.g., when a patient is expected to enter hypotension), an indication of an effectiveness of a treatment, and/or other aspect described herein.

The one or more processors 116 can be one or more hardware and/or electronic processors. The processor(s) 116 can include one or more of a microprocessor, a controller, a digital signal processor (DSP), an application specific integrated circuit (ASIC), a field-programmable gate array (FPGA), or other equivalent discrete or integrated logic circuitry. In some embodiments, the one or more processors 116 can include one or more graphical processing units (GPUs). The one or more GPUs may be configured to conduct linear algebraic calculations on matrices. For example, the one or more GPUs may be used by the machine learning model 124 to perform the operations described below.

The memory 120 can include computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). The memory 120 can include volatile and non-volatile computer-readable memories. Examples of volatile memories can include random access memories (RAM), dynamic random access memories (DRAM), static random-access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

In some embodiments, the hemodynamic sensing system 100 can include or be in communication with a remote computing device 140 via the network 136. The remote computing device 140 can include a computing system such as a client terminal at a hospital or clinic. The remote computing device 140 may include a computer in the ICU or in the OR. Additionally or alternatively, the remote computing device 140 may include a mobile electronic device, such as a laptop, a smartphone, a health monitor, and/or other electronic device. In some embodiments, the remote computing device 140 may include a display interface. The remote computing device 140 may be configured to receive (e.g., via the network 136) and/or display data transmitted from the hypotension analysis system 114, such as a determined time to event, the HPI, an indication of the effectiveness of a hypotension treatment, an indication of a type or characterization of a hypotension treatment, a recommendation as to how to modify and/or implement a hypotension treatment, one or more heart health parameters described herein, imagery associated with the heart health of the patient 104, and/or other data. The hemodynamic sensing system 100 may perform one or more actions described herein, such as determining a therapy protocol for the patient and/or generating a command to cause an infusion pump to deliver therapy to the patient 104. The network 136 can include a wireless and/or wired network connection.

### Hemodynamic Sensor

The hemodynamic sensor 108 can include one or more sensors coupled to (e.g., attached to, inserted within, etc.) the patient 104. The hemodynamic sensor 108 can obtain (e.g., receive, sense) a hemodynamic signal representative of an arterial pressure waveform of the patient 104 (see, e.g., the arterial pressure signal waveform 200). The sensed signal can be converted to data (e.g., digital data) by the signal converter 112.

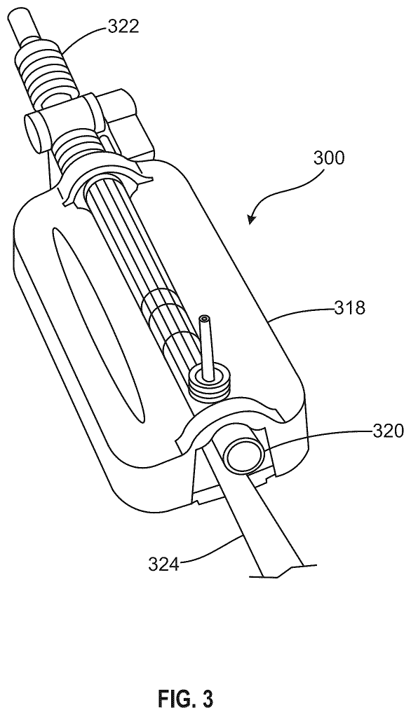

The hemodynamic sensor 108 can be inserted into the patient via a femoral arterial catheter inserted into a leg of the patient. Additionally or alternatively, the hemodynamic sensor 108 can include a minimally invasive hemodynamic sensor that can be attached to the patient via, e.g., a radial arterial catheter inserted into an arm of the patient (see, e.g., the hemodynamic sensor 300). For example, in some embodiments, the hemodynamic sensor 108 includes a non-invasive hemodynamic sensor that can be attached to the patient via one or more finger cuffs configured to sense data representative of arterial pressure of the patient. For example, the hemodynamic sensor 108 can include an inflatable finger cuff and a heart reference sensor (see, e.g., FIG. 3). In some embodiments, the hemodynamic sensor 108 does not include any invasive hemodynamic sensor (e.g., an in-line hemodynamic sensor, such as the hemodynamic sensor 300). In some embodiments, the hemodynamic sensor 108 includes a wireless (e.g., infrared) or wired connection to the signal converter 112.

The hemodynamic sensor 108 can take regular hemodynamic signal measurements of the patient 104. The measurements may take place about every 5 s, about every 10 s, about every 20 s, about every 30 s, about every 45 s, about every 1 min, about every 2 min, about every 5 min, about every 10 min, about every 15 min, about every 20 min, about every 30 min, about every 1 hour, any value therein, or fall within a range having endpoints therein. For example, in some embodiments the measurements are taken about every 15 minutes. The rate of measurement may be determined in part by the determined heart health of the patient, such as, for example, whether the patient 104 is currently in hypotension, what an estimated time to a hypotensive event is, an estimated degree of effectiveness of a hypotensive treatment is, whether one or more of the heart health parameters exceeds corresponding one or more thresholds, etc. The determined rate of measurement may be automatically determined or may be set by a user. While the hemodynamic sensor 108 can monitor the arterial pressure of the patient 104 over an extended period of time, the hemodynamic sensor 108 may only need to monitor the arterial pressure of patient 104 for a few minutes (e.g., 5 minutes) to generate enough data for the hypotension analysis system 114 to determine the estimated time to event and/or effectiveness of the treatment.

### Signal Converter

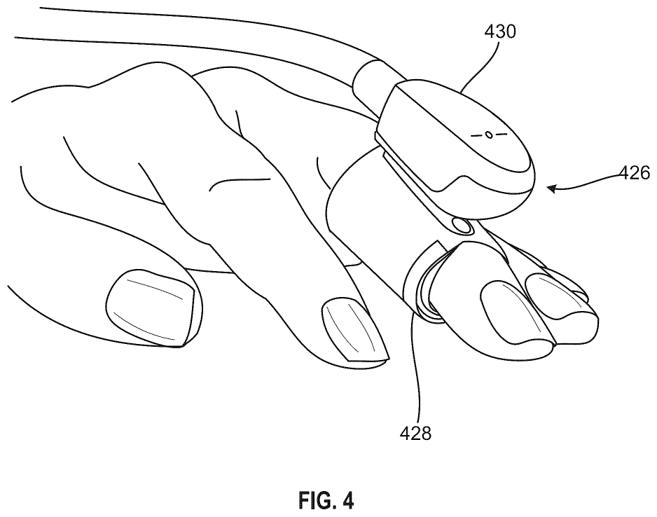

The signal converter 112 may include an analog-to-digital converter (ADC) and/or a digital-to-analog converter (DCA). The signal converter 112 may include a hardware and/or software converter. The signal converter 112 can transmit the converted data to the hypotension analysis system 114. Example signal converters 112 are described below with reference to FIGS. 3 and 4.

### Hypotension Analysis System

The hypotension analysis system 114 can receive the converted data from the signal converter 112 via the hemodynamic data interface 118. In some embodiments, the signal converter 112 is configured to convert the data to a form (e.g., format) that can be read and/or accepted by the hemodynamic data interface 118. Once the converted data is obtained by the hypotension analysis system 114, the processor 116 can execute instructions stored on the memory 120 to conduct analysis on the converted data.

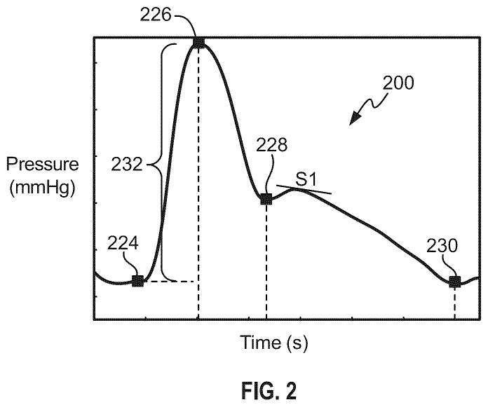

The converted data can comprise one or more health parameters, such as heart health parameters (see also FIG. 2). The heart health parameters may be highly predictive of potential (e.g., future) or actual (e.g., present) hypotension for the patient 104. These heart health parameters may be derived from the digital hemodynamic waveform data. The hemodynamic sensing system 100 can utilize some or all of the heart health parameters to predict whether hypotension will occur, assess an effectiveness of a hypotensive intervention, estimate a time to event (TTE) of hypotension, and/or generate a hypotension prediction index (hereinafter “HPI”) corresponding to the probability of a future hypotension event and/or an associated endotype therefor for the patient 104.

As described herein, one or more of the heart health parameters may be transmitted to the graphical user interface (GUI) 132 for display. The graphical user interface 132 can alert a user (e.g., a healthcare professional or patient himself or herself) about the determined TTE or treatment effectiveness and/or recommend an action item to modify an intervention. Such an alert can help ensure that a timely warning of a potential emergency hypotension event is provided to the user. Moreover, by enabling the user to access the graphical user interface 132 showing or displaying the one or more heart health parameters identified as indicative of present or future hypotension, the graphical user interface 132 can provide detailed diagnostic information allowing the user to identify a most probable cause of the lack of effectiveness of the hypotension intervention and/or best medical interventions for the prevention or treatment for the patient in real time.

### Machine Learning Model

With further reference to FIG. 1, the hypotension analysis system 114 can be configured to identify one or more of the heart health parameters relevant to the determination of the time to event and/or the determination of the effectiveness of an intervention. The heart health parameters can include health parameters that can be measured from the arterial pressure signal waveform and/or that may be useful in identifying (e.g., diagnosing) aspects of hypotension, such as the effectiveness of an intervention, whether an intervention has been undertaken, and/or an expected time to event (TTE) of hypotension. There are many potential heart health parameters that may be extracted from the arterial pressure signal waveform, but they can include, for example, cardiac output (CO), stroke volume (SV), stroke volume index (SVI), stroke volume variation (SVV), diastolic pressure (DIA), pulse rate (PR), heart rate (HR), stroke volume index (SVI), systemic vascular resistance (SVR), mean arterial pressure (MAP), average distance between subsequent MAPs, average distance between a systolic peak and a respective diastolic peak, HPI, time-based changes in arterial pressure, normalized area of pulse pressure, and/or others. Additionally or alternatively, the heart health parameters can include systemic vascular resistance index (SVRI), cardiac index (CI), and/or systolic pressure (SYS). a mean arterial pressure (MAP);

The hypotension analysis system 114 may receive the converted data (e.g., the heart health parameters) from the signal converter 112 via the hemodynamic data interface 118. The hypotension analysis system 114 may then transmit the heart health parameters to the machine learning model 124. The machine learning model 124 can be configured to receive the heart health parameters and encode them into different health parameters. As described in more detail below, the machine learning model 124 can include one or more layers, such as convoluted neural network (CNN) layers, long short-term memory (LSTM) layers or other recurrent neural network (RNN) layers, a transformer layer (e.g., a multi-head self-attention layer), one or more fully connected layers, an output layer, and/or other layers described herein.

The machine learning model 124 can include a fully connected deep learning model, a convoluted deep learning model, and/or some other type of learning model. In some embodiments, one or more of these heart health parameters and/or outputs based on the same can be transmitted to the graphical user interface 132 for display to a healthcare professional.

### Graphical User Interface

The graphical user interface 132 can provide a user interface that includes one or more control elements to enable user interaction and/or input therein. User input may be transmitted to the hypotension analysis system 114. The graphical user interface 132 may provide a sensory alarm based on measured and/or analyzed data from the arterial pressure signal waveform (e.g., from the one or more extracted heart health parameters), as described herein. The sensory alarm can be configured to provide a warning to medical personnel based on whether a hypotension predictive index (HPI) or other analysis raises an emergency. The sensory alarm may additionally or alternatively include instructions for how to treat a patient's possible hypotensive response, how to modify an existing treatment, when an expected time to event (TTE) of hypotension is expected to occur, the level of urgency of treatment, and/or relevant heart health parameters that should be addressed based on the analysis. The sensory alarm 158 can be implemented as one or more of a visual alarm, an audible alarm, a haptic alarm, and/or other type of sensory alarm. For example, the sensory alarm can be invoked as any combination of flashing and/or colored graphics shown by the graphical user interface 132. Additionally or alternatively, the graphical user interface 132 may display the determined TTE and/or effectiveness of an intervention via graphical user interface 132, a warning sound such as a siren or repeated tone, and a haptic alarm configured to cause a hemodynamic monitor (not shown) to vibrate or otherwise deliver a physical impulse perceptible to a medical worker or other user. The signal for the haptic alarm may be transmitted wirelessly via the network 136.

The graphical user interface 132 can include a liquid crystal display (LCD), a light-emitting diode (LED) display, an organic light-emitting diode (OLED) display, or other display device suitable for providing information to users in graphical form. The graphical user interface 132 can include one or more touch-sensitive and/or presence sensitive elements, such as a touch-sensitive display screen. In some embodiments, user input can be received in the form of gesture input, such as touch gestures, scroll gestures, zoom gestures, or other gesture input. In some examples, the graphical user interface 132 can include one or more physical control elements, such as a physical buttons, keys, knobs, mouse, keyboard, or other physical control elements configured to receive user input to interact with components of the hemodynamic sensing system 100. However, in some embodiments the graphical user interface 132 does not allow for user selection (e.g., does not take user input) but instead provides only output to a user.

### Pump

With further reference to FIG. 1, the hemodynamic sensing system 100 can include a pump 110 that can provide a therapy to the patient 104. The hypotension analysis system 114 can communicate with the pump 110 (e.g., via the data interface 118). The pump 110 can include an infusion pump. For example, the pump 110 can include a gravity infusion pump, a syringe infusion pump, an elastomeric infusion pump, a volumetric infusion pump, a patient-controlled analgesia (PCA) pump, an enteral infusion pump, an insulin pump, an ambulatory infusion pump, and/or any other kind of infusion pump.

A gravity infusion pump can use gravity to deliver fluids into the an intravenous (IV) line and ultimately into the patient's body. The infusion container may be located above the patient 104 for gravity to have its effect. A syringe infusion pump may be built in or directly connected to the pump system. A syringe infusion pump can provide small volume infusions and/or offer increased control over the fluid delivery.

An elastomeric infusion pump can be a single-use pump that functions without an external power source. Such a pump 110 can include one or more elastomeric balloons filled with fluid, which can create a positive pressure to push the fluid out along the IV line. In some embodiments, the pump 110 can allow the patient 104 to at least partially control the administration of therapy, such as in PCA pumps.

The pump 110 can receive instructions from the hypotension analysis system 114, such as via the data interface 118. The hypotension analysis system 114 can generate a control signal to instruct the pump 110 to deliver therapy to the patient 104, such as intravenous therapy using an intravenous therapeutic agent. The therapy provided may be based on an endotype determined by the hypotension analysis system 114. Additionally or alternatively, the therapy may be based on a therapeutic protocol, such as one determined by the hypotension analysis system 114. The therapeutic protocol may include instructions relating to a type (or types) of therapeutic agent(s), an amount of the therapeutic agent(s), a length of time associated with delivery of the therapeutic agent(s), and/or any other therapy described herein.

The therapy can include intravenous infusion of one or more fluids in order to expand plasma volume, which can help increase blood pressure. The type of fluid(s) used (e.g., crystalloid and/or colloid) can be based on the patient's condition (e.g., endotype and/or likelihood of developing that endotype) and the desired effect. The fluid therapy can correct a volume deficit and/or provide additional oxygen. Additionally or alternatively, the therapy may include one or more vasopressors, which can include medications used to constrict blood vessels. This can increase a vascular resistance and blood pressure. Vasopressors can stimulate receptors in the cardiovascular system to induce vasoconstriction. Additionally or alternatively, the pump 110 can deliver inotropes. These drugs enhance the contractility of the heart muscle, improving cardiac output. Inotropes can improve myocardial contractility and/or heart rate, which in turn may support better blood pressure control. In some embodiments, the therapy can include fluid resuscitation, which can increase blood volume and/or improve tissue perfusion. The therapeutic agents may be initially titrated using a lower dose, possibly with a ramped up dose over time, to reduce a likelihood of adverse effects.

### Arterial Pressure Signal Waveform

FIG. 2 is a graph illustrating an example arterial pressure signal waveform 200. The arterial pressure signal waveform 200 can include a plurality of attributes each indicative of one or more aspects of blood flow, cardiac output, and/or other aspects of heart health parameters described herein. The arterial pressure signal waveform 200 can correspond to a single heartbeat of the patient 104. The hemodynamic sensor 108 can be configured to detect new arterial pressure signal waveforms 200 for a plurality of heartbeats in a particular timeframe. For example, the hemodynamic sensor 108 may be able to generate a new arterial pressure signal waveform 200 for each heartbeat.

The arterial pressure signal waveform 200 is an example waveform that corresponds to hemodynamic data sensed by the hemodynamic sensor 108 and converted by the signal converter 112. Arterial pressure signal waveform 200 (represented via digital hemodynamic data) can include various indicia indicative of heart health for the patient 104. Various heart health parameters can be extracted by the hypotension analysis system 114, as discussed herein. Prior to extracting the heart health parameters, a beat detector can identify a start and an end of each heartbeat corresponding to each waveform. The beat detector can be a hardware detector (e.g., a resistance detector, an inductance detector, optical detector, etc.) or a software detector, such as a software beat detector. The beat detector may identify the start of the heartbeat based on a maximum arterial pressure, a minimum arterial pressure, a maximum or minimum rate of change in the arterial pressure, and/or a second derivative in the arterial pressure with respect to time in the arterial pressure. Based on the heartbeat identification within the arterial pressure signal waveform 200, various health parameters of heart health can be extracted from the waveform on an on-going (e.g., real-time), beat-to-beat basis. In some embodiments, the hypotension analysis system 114 can obtain any necessary input data from a single arterial pressure signal waveform 200 without needing to rely on a plurality of such arterial pressure signal waveforms.

The start indicator 224 of the arterial pressure signal waveform 200 corresponds to the start of a heartbeat. The systolic maximum indicator 226 of the arterial pressure signal waveform 200 corresponds to a maximum systolic pressure, marking an end of systolic rise. The notch indicator 228 of the arterial pressure signal waveform 200 corresponds to a presence and corresponding pressure of a dicrotic notch, marking an end of systolic decay. The diastolic minimum indicator 230 of the arterial pressure signal waveform 200 corresponds to a minimum diastolic pressure of the heartbeat of the patient 104. Further, arterial pressure gradients, or pressure differences between points of the arterial pressure signal waveform 200, can be used to identify the heart health parameters. For instance, a pulmonary pulse pressure 232 represents the difference between minimum diastolic pressure (the diastolic minimum indicator 230) and maximum systolic pressure (the systolic maximum indicator 226). As shown, slope S1 is a slope of the arterial pressure signal waveform 200, which may also be useful in determining the heart health parameters. The slope S1 is depicted at one location but is representative of multiple slopes that may be determined at multiple locations along arterial pressure signal waveform 200. For instance, a maximum and/or a minimum time derivative of the arterial pressure signal waveform 200 may be used to calculate the heart health parameters.

Additional indicators of the arterial pressure signal waveform 200 can be useful for calculating heart health parameters. For example, an interval between the systolic maximum indicator 226 and the notch indicator 228 can be extracted from the arterial pressure signal waveform 200. Additionally or alternatively, an interval between the start indicator 224 and the diastolic minimum indicator 230 can be extracted from the arterial pressure signal waveform 200. The hypotension analysis system 114 may use these and/or other indicators from the arterial pressure signal waveform 200 to identify additional heart health parameters from the arterial pressure signal waveform 200. For example, a systolic rise (e.g., between the start indicator 224 and the systolic maximum indicator 226), a systolic decay (e.g., between the systolic maximum indicator 226 and the notch indicator 228), a systolic phase (e.g., between the start indicator 224 and the notch indicator 228), a diastolic phase (e.g., between the notch indicator 228 and the diastolic minimum indicator 230), and/or a heartbeat interval (between successive start indicators 224) can be determined by the hypotension analysis system 114. Such indicia may include the mean arterial pressure during one of the above-referenced intervals. The area under the curve of arterial pressure signal waveform 200 and the standard deviations of the arterial pressure signal waveform 200 determined for the above-referenced intervals can also serve as heart health parameters and/or inputs for calculating the same.

### Example Hemodynamic Sensors

FIG. 3 is a perspective view of an example hemodynamic sensor 300 that can be coupled (e.g., attached) to the patient 104 for sensing hemodynamic data representative of arterial pressure of the patient. The hemodynamic sensor 108 of FIG. 1 may include the hemodynamic sensor 300 and/or include one or more features thereof. The hemodynamic sensor 300 is one example of a minimally invasive hemodynamic sensor that can be attached to the patient 104 via, e.g., a radial arterial catheter inserted into an arm of the patient. In other examples, the hemodynamic sensor 300 can be attached to the patient 104 via a femoral arterial catheter inserted into a leg of the patient.

As illustrated, the hemodynamic sensor 300 includes a housing 318, a fluid input port 320, a catheter-side fluid port 322, and an I/O cable 324. The fluid input port 320 is configured to be connected via tubing or other fluidic (e.g., hydraulic) connection to a fluid source, such as a saline bag or other fluid input source. The catheter-side fluid port 322 is configured to be connected via tubing or other fluidic connection to a catheter (e.g., a radial arterial catheter or a femoral arterial catheter) that is inserted into an arm of the patient (i.e., a radial arterial catheter) or a leg of the patient (i.e., a femoral arterial catheter). The I/O cable 324 may be configured to connect to a hemodynamic monitor (e.g., the graphical user interface 132) via, e.g., one or more of I/O connectors in the monitor. The housing 318 of the hemodynamic sensor 300 can contain (e.g., enclose) one or more pressure transducers, communication circuitry, processing circuitry, and/or corresponding electronic components to sense fluid pressure corresponding to an arterial pressure of the patient. One or more of these may be transmitted to the hemodynamic monitor (e.g., the graphical user interface 132) via the I/O cable 324.

In operation, a column of fluid (e.g., saline solution) can be introduced from a fluid source (e.g., a saline bag) through the hemodynamic sensor 300 via fluid input port 320 to catheter-side fluid port 322 toward the catheter inserted into the patient. The arterial pressure is communicated through the fluid column to pressure sensors located within housing 316 which sense the pressure of the fluid column. The hemodynamic sensor 300 translates the sensed pressure of the fluid column to an electrical (e.g., analog) signal via the pressure transducers and outputs the corresponding electrical signal. The hemodynamic sensor 300 can therefore transmit, to the hypotension analysis system 114 (e.g., via the signal converter 112), analog sensor data (or a digital representation of the analog sensor data) that is representative of real-time, perhaps even beat-to-beat, monitoring of the arterial pressure of the patient.

FIG. 4 is a perspective view of an example hemodynamic sensor 426 for sensing hemodynamic data representative of arterial pressure of the patient. The hemodynamic sensor 426 is an example of a non-invasive hemodynamic sensor that can be attached to the patient via one or more finger cuffs to sense data representative of arterial pressure of the patient. The hemodynamic sensor 426 includes an inflatable finger cuff 428 and a heart reference sensor 430. The inflatable finger cuff 428 can include an inflatable blood pressure bladder configured to inflate and deflate as controlled by a pressure controller (not illustrated) that may be pneumatically connected to the inflatable finger cuff 428. The inflatable finger cuff 428 can additionally or alternatively include an optical (e.g., infrared) transmitter and/or an optical receiver that are electrically connected to the pressure controller (not illustrated). The optical transmitter and the optical receiver can measure the changing volume of the arteries under the cuff in the finger. The optical transmitter and the optical receiver can be positioned to transmit and receive light therebetween through the inflatable blood pressure bladder.

In operation, the pressure controller may continually adjust pressure within the finger cuff to maintain a constant volume of the arteries in the finger (e.g., an unloaded volume of the arteries) as measured via the optical transmitter and optical receiver of inflatable finger cuff 428. The pressure applied by the pressure controller to continuously maintain the unloaded volume can be representative of the blood pressure in the finger and can be communicated by the pressure controller to the hypotension analysis system (e.g., the hypotension analysis system 114). The heart reference sensor 430 can measure the hydrostatic height difference between the level at which the finger is kept and a reference level for the pressure measurement, which typically is a heart level. Accordingly, the hemodynamic sensor 426 transmits sensor data that is representative of substantially continuous beat-to-beat monitoring of the arterial pressure waveform of the patient. As noted above, this sensor data can be used to extract an arterial pressure signal waveform 200 (e.g., the arterial pressure signal waveform 200) and/or one or more heart health parameters therefrom.

### Deep Learning Model

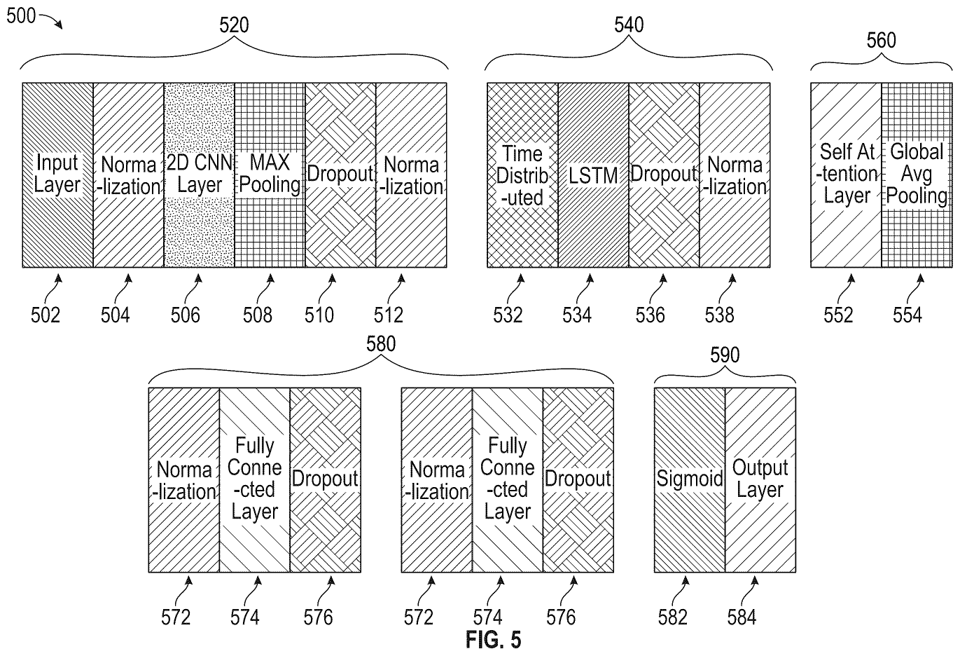

FIG. 5 shows an example architecture of a deep learning model 500 that may be used, for example, by the machine learning model 124. The hypotension analysis system 114 can use the deep learning model 500 to output an estimated time to event (TTE), a level of effectiveness of an intervention (and/or whether an intervention has been implemented), and/or a hypotension prediction index (HPI). The deep learning model 500 may use one or more of the heart health parameters that are relevant to identification details related to hypotension to help determine the TTE, the effectiveness of the intervention, and/or. The heart health parameters can include health parameters that can be measured from the arterial pressure signal waveform (e.g., the arterial pressure signal waveform 200) and/or that may be useful in identifying (e.g., diagnosing) hypotension or some aspect related thereto. There are many potential heart health parameters that may be extracted from the arterial pressure signal waveform, but they can include, for example, cardiac output (CO), stroke volume (SV), stroke volume index (SVI), stroke volume variation (SVV), diastolic pressure (DIA), pulse rate (PR), heart rate (HR), stroke volume index (SVI), systemic vascular resistance (SVR), mean arterial pressure (MAP), average distance between subsequent MAPs, average distance between a systolic peak and a respective diastolic peak, HPI, time-based changes in arterial pressure, normalized area of pulse pressure, and/or others.

The hypotension analysis system 114 may receive the converted data (e.g., the heart health parameters in digital form) from the signal converter 112 via the hemodynamic data interface 118. The hypotension analysis system 114 may then transmit the heart health parameters to the machine learning model 124, which may include the deep learning model 500.

The deep learning model 500 can take the parameters and pass them through one or more networks. Each network may include one or more constituent layers. For example, as shown in FIG. 5, the deep learning model 500 can include a convoluted neural network (CNN) 520, a long short-term memory network 540, a transformer 560, one or more fully connected networks 580, and/or an output network 590.

The convoluted neural network 520 can include an initial input layer 502, a first normalization layer 504, a two-dimensional CNN layer 506, a maximum pooling layer 508, a dropout layer 510, and/or a second normalization layer 512. The input layer 502 can be the initial layer of the network where the raw input data (e.g., the heart health parameters) is fed. The heart health parameters may be passed through the input layer 502 as images and/or time-series data.

The input layer 502 can include a grid of neurons. Each neuron can correspond to a pixel in the input image or a feature of the time-series data. Each neuron of the input layer 502 can be connected to a small local region of the input data. This local region can correspond to a small patch of pixels or data points in the image or time series. Each neuron can apply a convolution operation to its local region, combining the input values corresponding weights of to produce a single output value. Accordingly, the input layer 502 can help the convoluted neural network 520 capture local patterns and features in the input data (e.g., heart health parameters). In some embodiments, the convoluted neural network 520 can apply an activation function, such as a Rectified Linear Unit (ReLU) is applied to the output of each neuron from the input layer 502 to introduce non-linearity into the network to enhance pattern recognition.

The local patterns may be normalized by the first normalization layer 504. The normalization layer 504 can standardize the resulting outputs from the input layer 502. The convoluted neural network 520 may include one or more normalization techniques, such as batch normalization and/or some other technique. Batch normalization can normalize a subset of data and/or normalize activations across each feature map independently. Normalizing the data can stabilize training by reducing internal covariate shifts, allow for faster convergence, providing noise that can prevent overfitting, and avoid problems like exploding or vanishing gradients.

The two-dimensional CNN layer 506 may function similarly to the input layer 502 in that can convolve data sets by connecting neurons of data to different aspects of the data, which may be image data for the two-dimensional CNN layer 506. The two-dimensional CNN layer 506 can generate one or more feature maps of the data, which can include corresponding two-dimensional representations of the data.

At the maximum pooling layer 508, the convoluted neural network 520 can reduce the spatial dimensions (e.g., width and/or height) of the input data. For example, the maximum pooling layer 508 can extract a maximum value within a certain subregion of the data (e.g., a 2×2 window) and remove the other data. Accordingly, the system can reduce computational complexity and/or reduce overfitting. Additionally or alternatively, the maximum pooling layer 508 can help extract dominant or clear features from the data. In some embodiments, an average pooling layer may be used additionally or alternatively to the maximum pooling layer 508.

The dropout layer 510 can regularize the output data by randomly setting a fraction of the neurons in a layer to zero (e.g., during training). Accordingly, these neurons can be temporarily dropped out of the network. Dropping neurons can reduce the interdependence between or among neurons and avoid overreliance on specific features and thus less likely to overfit to the training data. During training, the neurons may be dropped. Additionally or alternatively, during inference of test data, neurons may not necessarily be dropped but weights of neurons can be scaled a compensation factor to compensate for the fact that more neurons may be active during inference than during training.

Dropping out neurons can introduce noise to the network during training and thus reduce fitting to noise present in the training data. This encourages the network to learn more robust features that are generalizable to unseen data.

The normalization layer 512 may perform many of the features of the normalization layer 504. For example, the normalization layer 512 can standardize the outputs from the dropout layer 510. In some embodiments, the resulting normalized data can be used as inputs in the long short-term memory network 540.

In some embodiments, the deep learning model 500 can include a recurrent neural network (RNN). For example, the deep learning model 500 can include one or more layers from a long short-term memory network 540, each of which apply their layer operations independently to each time step of a sequence of the input data from the heart health parameters. For example, the time distributed layer 532 (or TimeDistributed layer) can be part of a long short-term memory network 540.

The long short-term memory network 540 can receive a sequence of data points with a temporal dimension, represented as a 3D tensor. These may be the output data, for example, from the convoluted neural network 520. The time distributed layer 532 can wrap around each layer of the long short-term memory network 540 (and/or of other layers in the deep learning model 500) at each time step. Each wrapped layer (e.g., dense layer) can be applied independently. The same set of weights may be used for each time step. The output of the time distributed layer 532 may be a sequence of outputs corresponding to the result of the wrapped layer to the corresponding time step of the input sequence.

The long short-term memory layer 534 can capture long-range dependencies in the output sequential data, such as by maintaining a memory state across time steps. The long short-term memory layer 534 can include memory cells that allow the long short-term memory network 540 to remember information over long sequences. These memory cells may maintain a hidden state (e.g., a cell state), which may be updated and/or passed along to subsequent time steps. The long short-term memory layer 534 can control the flow of information using one or more gates, such as a forget gate, an input, and/or an output gate. A forget gate can determine which information to discard from the cell state. An input gate can determine which new information to store in the cell state. Additionally or alternatively, an output gate can determine which information to output based on the current input and the cell state.

At each time step, the long short-term memory layer 534 can generate update algorithms based on the input at the current time step, the previous hidden state, and/or the previous cell state. These update algorithms can determine how to update the cell state and the hidden state. The long short-term memory layer 534 may apply one or more non-linear transformations to the input and the hidden state, such as the sigmoid and hyperbolic tangent (tanh) functions. These functions can help control the flow of information through the gates and to update the cell state.

During training, the long short-term memory layer 534 can compute gradients and/or update parameters using backpropagation through time (BPTT). The long short-term memory layer 534 can use BPTT over multiple time steps to learn the relationships between inputs at different time steps. In this way, the long short-term memory layer 534 can capture long-range dependencies and remember information over extended sequences.

The dropout layer 536 may include one or more features of the dropout layer 510. For example, the dropout layer 536 may randomly set a portion of the neurons in a layer to zero during training. Additionally or alternatively, during inference the dropout layer 510 can modify the weights of neurons. The normalization layer 538 can include one or more features of the normalization layer 504 and/or the normalization layer 512. For example, the normalization layer 538 may standardize the outputs from the dropout layer 536. Resulting normalized data can be used as inputs in the transformer 560.

The transformer 560 can capture long-range dependencies in the received data from the long short-term memory network 540. Unlike the convoluted neural network 520 and the long short-term memory network 540, which process their input data sequentially or hierarchically, the transformer 560 include a self-attention mechanism. Self-attention allows the transformer 560 to weigh the importance of different words or tokens in a sequence of the received input data when processing each token. This mechanism allows the transformer 560 to more fully capture contextual relationships among tokens, regardless of their position in the sequence.

The transformer 560 can include an encoder-decoder architecture. The encoder can process the input sequence. Additionally or alternatively, the decoder can generate an output sequence. In some embodiments, both the encoder and decoder include one or more layers of self-attention mechanisms and feed-forward neural networks.

A multi-head self-attention layer 552 can include one or more such multi-head self-attention layers. The multi-head self-attention layer 552 can implement multi-head attention, which can include running multiple parallel self-attention operations. Each operation can focus on different aspects of the input sequence. Outputs from these parallel attention heads can be concatenated and/or linearly transformed to produce a final attention output. Positional encoding can be added to the input data to provide the deep learning model 500 with information about the position of each token in the sequence.

In some embodiments, the transformer 560 can include a feed-forward neural networks (FFNs) in one or more of the multi-head self-attention layer(s) 552. These FFNs can include one or more fully connected layers with a non-linear activation function, such as the ReLU Rectified Linear Unit (ReLU) function.

The global average pooling layer 554 can include one or more features of the maximum pooling layer 508. In some embodiments, the global average pooling layer 554 pools the layers using an average rather than pooling by a maximum value. For example, the global average pooling layer 554 can extract an average value within a region the data (e.g., a 2×2 window). In some embodiments, the maximum pooling layer 508 can remove the other data.

The fully connected networks 580 can be included as part of the transformer 560 described above. For example, the fully connected networks 580 may include the FFNs described above. Additionally or alternatively, the fully connected networks 580 can include additional features.

The fully connected networks 580 can include one or more normalization layers 572 to normalize the data, one or more fully connected layers 574, and/or one or more dropout layers 576. The normalization layers 572 may include one or more features of the normalization layer 504 and/or the normalization layer 538. The dropout layers 576 may include one or more features of the dropout layer 510 and/or the dropout layer 536.

The fully connected layers 574 can include one or more encoding filters that can perform mathematical operations to transform the input data into compressed data. Data samples of each element (e.g., heart health parameter or modified versions thereof) of the input data can be reduced by a target encoding factor by each of the encoding filters until fully compressed data reaches a latent space. Decoding filters can be applied to the compressed data in the latent space to yield output data. Data samples may be expanded by a target decoding factor (e.g., different from the target encoding factor) by each of the decoding filters until resulting in the output data. In some embodiments, an autoencoder model can autogenerate the architecture of the encoding filters and/or the decoding filters.

The fully connected deep learning autoencoder model may be part of a feed-forward neural network (FFN) described above. In the fully connected networks 580, each node in one layer may be connected to every node in the subsequent layer. Additionally or alternatively, the output from each node can serve as an input to every node in the subsequent layer. Each node can represent a corresponding input value, such as a heart health parameter or a modified feature thereof. The fully connected networks 580 can include one or more hidden layers between the input data and the latent space. Each layer can apply a weight or weighting to data for each node. This weight may be learned during a training phase of the model. Each hidden layer may apply one or more activation functions to the received interim data from the precedent layer. One or more of the activation functions may be a non-linear function, such as a trigonometric function. In some embodiments, the activation function includes a hyperbolic tangent (tanh) function.

During training, weights of each connection between layers (and/or of the activation functions and/or values thereof themselves) may be adjusted by backpropagating and/or gradient descent. The fully connected networks 580 can adjust these connection weights based on a goal of reducing or even minimizing a difference between a predicted output and an actual output.

The deep learning model 500 can include an output network 590 in some embodiments. The output network 590 can include a sigmoid layer 582 or some other non-linear function. The sigmoid layer 582 can include an activation function to map any input value to a value between 0 and 1. The sigmoid layer 582 can introduce non-linearity into the output of the deep learning model 500. Introducing non-linearity can improve the ability of the deep learning model 500 to learn complex patterns and relationships in the data.

The sigmoid function can include the function:

$$σ ( x ) = \frac{1}{1 + {e}^{- x}}$$

The sigmoid layer 582 can modify each element of the output layer 584. The output of the output layer 584 can be interpreted as probabilities of generating specific values for each feature or pixel in the generated data. This output can include the hypotension prediction index (HPI) described herein, or a slightly modified version thereof.

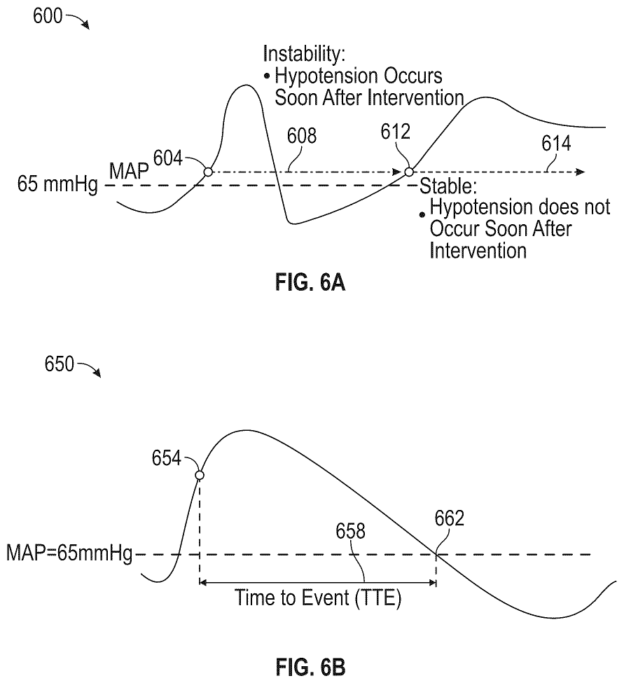

FIG. 6A shows an example graph 600 of a patient's mean arterial pressure (MAP) over time. The graph 600 illustrates how the deep learning model 500 described above can help identify a instability start point 604 and help introduce stability of a patient's blood pressure at a instability end point 612. The deep learning model 500 can help identify whether an intervention is reducing or increasing stability within the instability duration 608 and/or during a stability duration 614. Using the heart health parameters and/or other inputs, the deep learning model 500 can reduce a length of the instability duration 608 and increase the length of the instability end point 612.

FIG. 6B shows a graph 650 of a patient's MAP over time. The deep learning model 500 can help identify from a start point 654 a time to event (TTE) 658. The time to event 658 can be defined as an expected time that a patient will enter hypotension at a hypotension begin point 662. Thus, the time to event 658 is defined as the time from the start point 654 to the hypotension begin point 662. As noted previously, hypotension may be defined as a MAP below 65 mmHg.

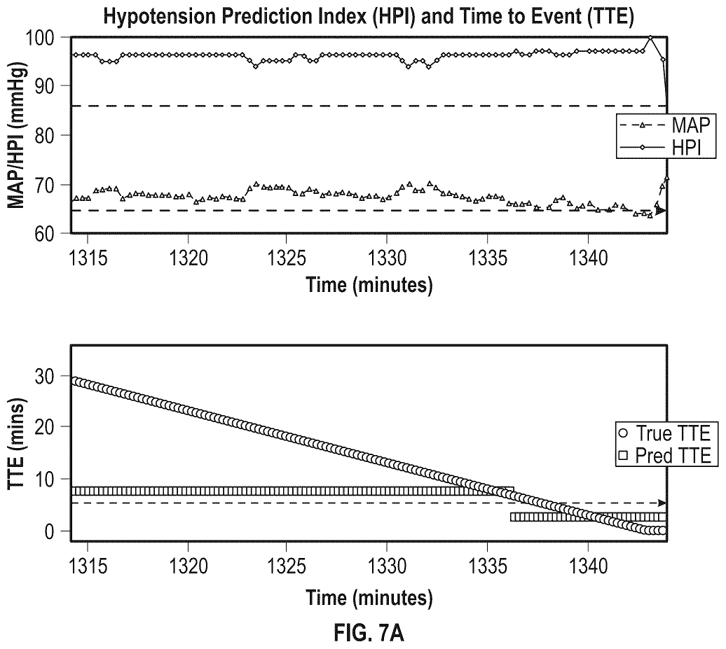

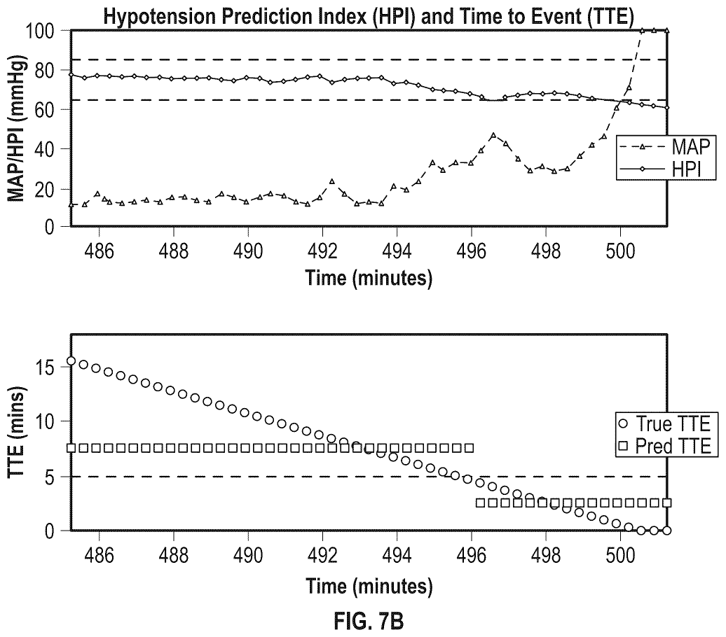

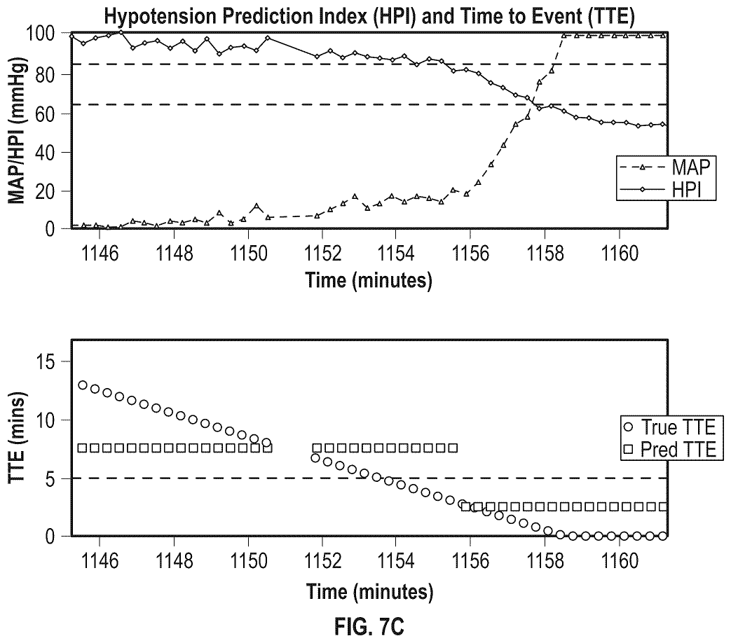

FIGS. 7A-7C show relationships between a determined HPI and the TTE over time, as determined by various embodiments of the systems and deep learning models described herein. In general, as the HPI drops below a threshold level (e.g., 85), this may indicate a short TTE (e.g., zero seconds). FIG. 7A shows the actual time of the event (e.g., TTE=zero) to occur around 1344 minutes, just before the HPI drops below the threshold level. The 7- and 2-minute TTE predictions are somewhat early. FIG. 7B shows the time of the hypotensive event at about the same time as the HPI going above the threshold level. FIG. 7B also shows an early 2-minute TTE prediction and a late 7-minute TTE prediction. FIG. 7C shows the results of an improved deep learning model and system compared to FIGS. 7A-7B. The TTE predictions are more accurate, with the 7-minute TTE a little late but the 2-minute TTE almost exact. Thus, the systems described herein can accurately predict a TTE and provide critical time to caregivers to take actions to prevent life-threatening drops in blood pressure from permanently harming a patient.

### Example Methods of Hypotension Analysis

The example routines or methods herein illustrate various implementations of systems described herein. The blocks of the routines illustrate example implementations, and in various other implementations various blocks may be rearranged, and/or rendered optional. Further, various blocks may be omitted from and/or added to the example routines below, and blocks may be moved between the various example routines.

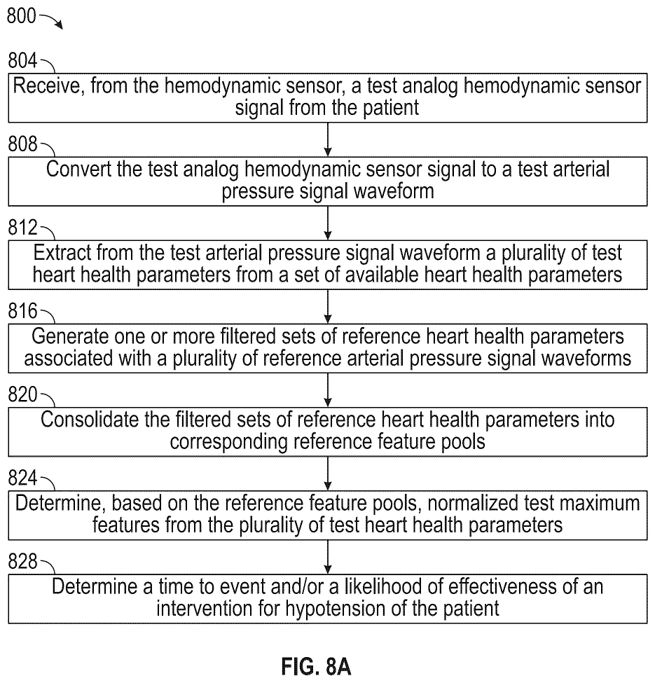

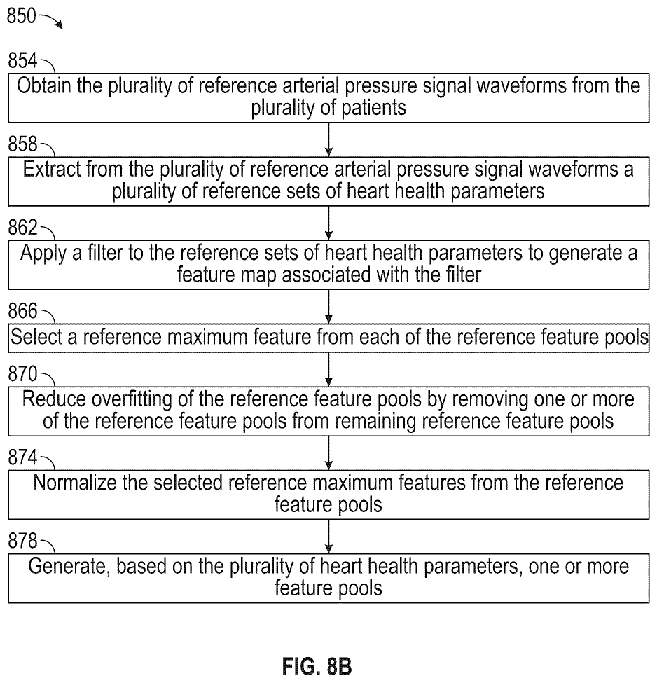

FIG. 8A shows a flowchart of an example method 800 for determining a time to event (TTE) and/or a likelihood of effectiveness of an intervention of hypotension. The method 800 can be performed by one or more systems described herein (e.g., the hemodynamic sensing system 100, the hypotension analysis system 114, the machine learning model 124, the deep learning model 500, etc.) and/or a combination thereof. At block 804, the system can receive, from a hemodynamic sensor, an analog hemodynamic sensor signal from a patient. The analog hemodynamic sensor signal may correspond to a signal sensed by a hemodynamic sensor (e.g., hemodynamic sensor 108, the hemodynamic sensor 300, the hemodynamic sensor 426).

At block 808, the system can convert the analog hemodynamic sensor signal to an arterial pressure signal waveform. The arterial pressure signal waveform can correspond to one described herein, such as the arterial pressure signal waveform 200. At block 812, the system can extract, from the arterial pressure signal waveform, a plurality of heart health parameters. The heart health parameters can correspond to any heart health parameters described herein, such as, for example, cardiac output (CO), stroke volume (SV), stroke volume variation (SVV), diastolic pressure (DIA), pulse rate (PR), stroke volume index (SVI), systemic vascular resistance (SVR), mean arterial pressure (MAP), HPI, systemic vascular resistance index (SVRI), cardiac index (CI), systolic pressure (SYS), and/or any others described herein. In some embodiments, the system may extract a combination of SVI, HR, CI, SVRI, and SVV from the arterial pressure signal waveform, but other combinations are possible.

At block 816, the system can generate, using a deep learning model (e.g., the deep learning model 500), one or more filtered sets of reference heart health parameters associated with a plurality of reference arterial pressure waveforms. In some embodiments, the method 800 can include obtaining the plurality of reference arterial pressure signal waveforms from a plurality of patients and extracting from the plurality of reference arterial pressure signal waveforms a plurality of reference sets of heart health parameters. Each of the reference sets of heart health parameters can include corresponding heart health parameters from the set of available heart health parameters.

At block 820, the system can consolidate the filtered sets of reference heart health parameters into corresponding reference feature pools. At block 824, the system can determine, based on the reference feature pools, normalized test features (e.g., maximum features) from the plurality of test heart health parameters.

At block 828, the system can determine, based on the test features from the from the plurality of heart health parameters, a time to event and/or a likelihood of effectiveness of an intervention for hypotension.

Additionally or alternatively the method 800 can include applying a filter to the reference sets of heart health parameters to generate a feature map associated with the filter.

In some embodiments, the method 800 can include selecting a reference feature (e.g., maximum feature, average feature) from each of the reference feature pools. Additionally or alternatively, the system can reduce overfitting of the reference feature pools by removing one or more of the reference feature pools from remaining reference feature pools. The system may normalize the selected reference maximum features from the reference feature pools and generate, based on the plurality of heart health parameters, one or more feature pools.

In some embodiments, the method 800 includes displaying the alert indicating the determined likelihood of effectiveness of the intervention and/or an alert indicating a determined time to event.

In some embodiments, the method 800 can include generating, based on the determined likelihood of effectiveness of the intervention, data for displaying at least the alert indicating the likelihood of effectiveness of the intervention for hypotension. The alert may include one or more of a symbol, a numerical value, a visual design, a repeated indicator, and/or a highlight.

In some embodiments, the method 800 includes determining, based on the comparison between the normalized selected reference maximum features and the normalized test maximum features, that the patient will experience hypotension within a specified time with at least a target threshold confidence level. Additionally or alternatively, the system may generate, based on the determination that that the patient will experience hypotension within the specified time with at least the target threshold confidence level, data for displaying, via the graphical user interface, the alert indicating the expected time to event of hypotension within the patient.

FIG. 8B shows a flowchart of an example method 850 for generating one or more feature pools. The method 850 can be performed by one or more systems described herein (e.g., the hemodynamic sensing system 100, the hypotension analysis system 114, the machine learning model 124, the deep learning model 500, etc.) and/or a combination thereof.

At block 854 the system can receive a plurality of reference hemodynamic sensor signals from a plurality of reference patients. At block 858 the system can extract from the reference hemodynamic sensor signals corresponding sets of reference heart health parameters. The reference heart health parameters may include one or more heart health parameters described above. At block 862 the system can apply a filter to the reference sets of heart health parameters to generate a feature map associated with the filter. The reference sets of heart health parameters may be used by the system at block 866 to select a reference feature (e.g., maximum feature, average feature) from each of the reference feature pools.

At block 870, the system can reduce overfitting of the reference feature pools by removing one or more of the reference feature pools from remaining reference feature pools. At block 874, the system can normalize the selected reference maximum features from the reference feature pools and, at block 878, generate one or more feature pools based on the plurality of heart health parameters.

In some embodiments, the system comprises various features that are present as single features (as opposed to multiple features). For example, in one embodiment, the system includes a single hemodynamic sensor 108 with a single signal converter 112, a single processor 116 and a single memory 120, a single machine learning model 124, as described herein. Multiple features or components are provided in alternate embodiments.

In some embodiments, the system (e.g., the hemodynamic sensing system 100) comprises one or more of the following: means for sensing heart pressure (e.g., the hemodynamic sensor 108, the hemodynamic sensor 300, the hemodynamic sensor 426), means for converting sensed data (e.g., the signal converter 112), and/or means for displaying data (e.g., the graphical user interface 132).

### Additional Example Implementations and Details Related to Computing Systems

In some implementations the systems described herein may comprise, or be implemented in, a “virtual computing environment”. As used herein, the term “virtual computing environment” should be construed broadly to include, for example, computer-readable program instructions executed by one or more processors to implement one or more aspects of the modules and/or functionality described herein. Further, in this implementation, one or more services/modules/engines and/or the like of the system may be understood as comprising one or more rules engines of the virtual computing environment that, in response to inputs received by the virtual computing environment, execute rules and/or other program instructions to modify operation of the virtual computing environment. For example, a request received from a user computing device may be understood as modifying operation of the virtual computing environment to cause the request access to a resource from the system. Such functionality may comprise a modification of the operation of the virtual computing environment in response to inputs and according to various rules. Other functionality implemented by the virtual computing environment (as described throughout this disclosure) may further comprise modifications of the operation of the virtual computing environment, for example, the operation of the virtual computing environment may change depending on the information gathered by the system. Initial operation of the virtual computing environment may be understood as an establishment of the virtual computing environment. In some implementations the virtual computing environment may comprise one or more virtual machines, containers, and/or other types of emulations of computing systems or environments. In some implementations the virtual computing environment may comprise a hosted computing environment that includes a collection of physical computing resources that may be remotely accessible and may be rapidly provisioned as needed (commonly referred to as “cloud” computing environment).

Implementing one or more aspects of the system as a virtual computing environment may advantageously enable executing different aspects or modules of the system on different computing devices or processors, which may increase the scalability of the system. Implementing one or more aspects of the system as a virtual computing environment may further advantageously enable sandboxing various aspects, data, or services/modules of the system from one another, which may increase security of the system by preventing, e.g., malicious intrusion into the system from spreading. Implementing one or more aspects of the system as a virtual computing environment may further advantageously enable parallel execution of various aspects or modules of the system, which may increase the scalability of the system. Implementing one or more aspects of the system as a virtual computing environment may further advantageously enable rapid provisioning (or de-provisioning) of computing resources to the system, which may increase scalability of the system by, e.g., expanding computing resources available to the system or duplicating operation of the system on multiple computing resources. For example, the system may be used by thousands, hundreds of thousands, or even millions of users simultaneously, and many megabytes, gigabytes, or terabytes (or more) of data may be transferred or processed by the system, and scalability of the system may enable such operation in an efficient and/or uninterrupted manner.

Various implementations of the present disclosure may be a system, a method, and/or a computer program product at any possible technical detail level of integration. The computer program product may include a computer-readable storage medium (or mediums) having computer-readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer-readable storage medium (or mediums). Computer-readable storage mediums may also be referred to herein as computer-readable storage or computer-readable storage devices.

The computer-readable storage medium can be a tangible device that can retain and store data and/or instructions for use by an instruction execution device. The computer-readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer-readable program instructions may be callable from other instructions or from itself, and/or may be invoked in response to detected events or interrupts. Computer-readable program instructions configured for execution on computing devices may be provided on a computer-readable storage medium, and/or as a digital download (and may be originally stored in a compressed or installable format that requires installation, decompression, or decryption prior to execution) that may then be stored on a computer-readable storage medium. Such computer-readable program instructions may be stored, partially or fully, on a memory device (e.g., a computer-readable storage medium) of the executing computing device, for execution by the computing device. The computer-readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some implementations, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to implementations of the disclosure. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart(s) and/or block diagram(s) block or blocks.

The computer-readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowcharts and block diagrams in the Figures illustrate the architecture, functionality, and operation of possible implementations of systems, methods, and computer program products according to various implementations of the present disclosure. In this regard, each block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted or optional in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, elements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, and/or the like with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above implementations may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows 11, Windows Server, and/or the like), Windows CE, Unix, Linux, SunOS, Solaris, Blackberry OS, VxWorks, or other suitable operating systems. In other implementations, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

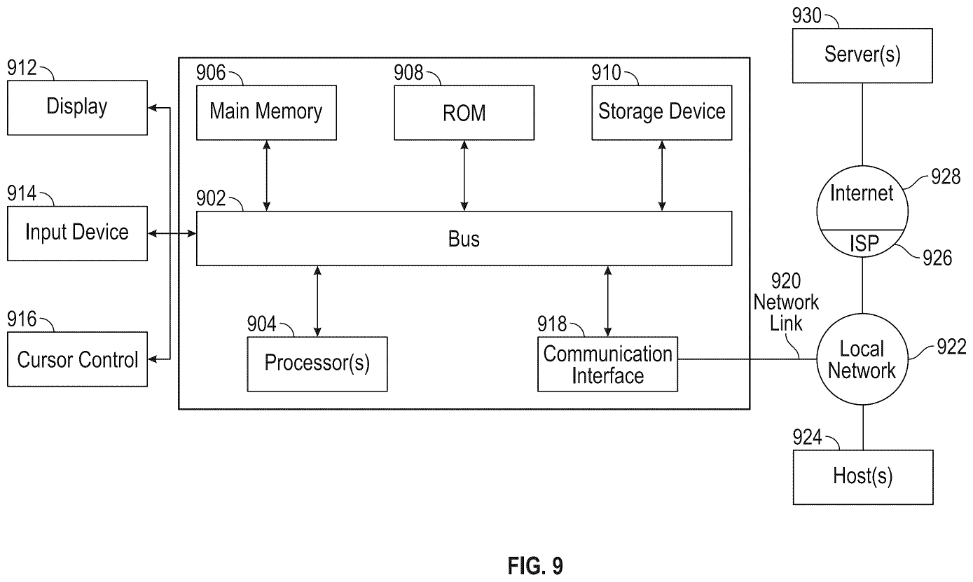

For example, FIG. 9 is a block diagram that illustrates a computer system 900 upon which various implementations may be implemented. For example, the computer system 900 may be implemented as the hemodynamic sensing system 100 (FIG. 1) in some implementations. Computer system 900 includes a bus 902 or other communication mechanism for communicating information, and a hardware processor, or multiple processors, 904 coupled with bus 902 for processing information. Hardware processor(s) 904 may be, for example, one or more general or special purpose microprocessors.

Computer system 900 also includes a main memory 906, such as a random access memory (RAM), cache and/or other dynamic storage devices, coupled to bus 902 for storing information and instructions to be executed by processor 904. Main memory 906 also may be used for storing temporary variables or other intermediate information during execution of instructions to be executed by processor 904. Such instructions, when stored in storage media accessible to processor 904, render computer system 900 into a special-purpose machine that is customized to perform the operations specified in the instructions.

Computer system 900 further includes a read only memory (ROM) 908 or other static storage device coupled to bus 902 for storing static information and instructions for processor 904. A storage device 910, such as a magnetic disk, optical disk, or USB thumb drive (Flash drive), etc., is provided and coupled to bus 902 for storing information and instructions.

Computer system 900 may be coupled via bus 902 to a display 912, such as a cathode ray tube (CRT) or LCD display (or touch screen), for displaying information to a computer user. An input device 914, including alphanumeric and other keys, is coupled to bus 902 for communicating information and command selections to processor 904. Another type of user input device is cursor control 916, such as a mouse, a trackball, or cursor direction keys for communicating direction information and command selections to processor 904 and for controlling cursor movement on display 912. This input device typically has two degrees of freedom in two axes, a first axis (e.g., x) and a second axis (e.g., y), that allows the device to specify positions in a plane. In some implementations, the same direction information and command selections as cursor control may be implemented via receiving touches on a touch screen without a cursor.

Computing system 900 may include a user interface module to implement a GUI that may be stored in a mass storage device as computer executable program instructions that are executed by the computing device(s). Computer system 900 may further, as described below, implement the techniques described herein using customized hard-wired logic, one or more ASICs or FPGAs, firmware and/or program logic which in combination with the computer system causes or programs computer system 900 to be a special-purpose machine. According to one implementation, the techniques herein are performed by computer system 900 in response to processor(s) 904 executing one or more sequences of one or more computer readable program instructions contained in main memory 906. Such instructions may be read into main memory 906 from another storage medium, such as storage device 910. Execution of the sequences of instructions contained in main memory 906 causes processor(s) 904 to perform the process steps described herein. In alternative implementations, hard-wired circuitry may be used in place of or in combination with software instructions.

Various forms of computer readable storage media may be involved in carrying one or more sequences of one or more computer readable program instructions to processor 904 for execution. For example, the instructions may initially be carried on a magnetic disk or solid state drive of a remote computer. The remote computer can load the instructions into its dynamic memory and send the instructions over a telephone line using a modem. A modem local to computer system 900 can receive the data on the telephone line and use an infra-red transmitter to convert the data to an infra-red signal. An infra-red detector can receive the data carried in the infra-red signal and appropriate circuitry can place the data on bus 902. Bus 902 carries the data to main memory 906, from which processor 904 retrieves and executes the instructions. The instructions received by main memory 906 may optionally be stored on storage device 910 either before or after execution by processor 904.

Computer system 900 also includes a communication interface 918 coupled to bus 902. Communication interface 918 provides a two-way data communication coupling to a network link 920 that is connected to a local network 922. For example, communication interface 918 may be an integrated services digital network (ISDN) card, cable modem, satellite modem, or a modem to provide a data communication connection to a corresponding type of telephone line. As another example, communication interface 918 may be a local area network (LAN) card to provide a data communication connection to a compatible LAN (or WAN component to communicated with a WAN). Wireless links may also be implemented. In any such implementation, communication interface 918 sends and receives electrical, electromagnetic or optical signals that carry digital data streams representing various types of information.

Network link 920 typically provides data communication through one or more networks to other data devices. For example, network link 920 may provide a connection through local network 922 to a host computer 924 or to data equipment operated by an Internet Service Provider (ISP) 926. ISP 926 in turn provides data communication services through the world wide packet data communication network now commonly referred to as the “Internet” 928. Local network 922 and Internet 928 both use electrical, electromagnetic or optical signals that carry digital data streams. The signals through the various networks and the signals on network link 920 and through communication interface 918, which carry the digital data to and from computer system 900, are example forms of transmission media.

Computer system 900 can send messages and receive data, including program code, through the network(s), network link 920 and communication interface 918. In the Internet example, a server 930 might transmit a requested code for an application program through Internet 928, ISP 926, local network 922 and communication interface 918.

The received code may be executed by processor 904 as it is received, and/or stored in storage device 910, or other non-volatile storage for later execution.

As described above, in various implementations certain functionality may be accessible by a user through a web-based viewer (such as a web browser), or other suitable software program). In such implementations, the user interface may be generated by a server computing system and transmitted to a web browser of the user (e.g., running on the user's computing system). Alternatively, data (e.g., user interface data) necessary for generating the user interface may be provided by the server computing system to the browser, where the user interface may be generated (e.g., the user interface data may be executed by a browser accessing a web service and may be configured to render the user interfaces based on the user interface data). The user may then interact with the user interface through the web-browser. User interfaces of certain implementations may be accessible through one or more dedicated software applications. In certain implementations, one or more of the computing devices and/or systems of the disclosure may include mobile computing devices, and user interfaces may be accessible through such mobile computing devices (for example, smartphones and/or tablets).

Many variations and modifications may be made to the above-described implementations, the elements of which are to be understood as being among other acceptable examples. All such modifications and variations are intended to be included herein within the scope of this disclosure. The foregoing description details certain implementations. It will be appreciated, however, that no matter how detailed the foregoing appears in text, the systems and methods can be practiced in many ways. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Conditional language, such as, among others, “can,” “could,” “might,” or “may,” unless specifically stated otherwise, or otherwise understood within the context as used, is generally intended to convey that certain implementations include, while other implementations do not include, certain features, elements, and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation.

The term “substantially” when used in conjunction with the term “real-time” forms a phrase that will be readily understood by a person of ordinary skill in the art. For example, it is readily understood that such language will include speeds in which no or little delay or waiting is discernible, or where such delay is sufficiently short so as not to be disruptive, irritating, or otherwise vexing to a user.

Conjunctive language such as the phrase “at least one of X, Y, and Z,” or “at least one of X, Y, or Z,” unless specifically stated otherwise, is to be understood with the context as used in general to convey that an item, term, etc. may be either X, Y, or Z, or a combination thereof. For example, the term “or” is used in its inclusive sense (and not in its exclusive sense) so that when used, for example, to connect a list of elements, the term “or” means one, some, or all of the elements in the list. Thus, such conjunctive language is not generally intended to imply that certain implementations require at least one of X, at least one of Y, and at least one of Z to each be present.

The term “a” as used herein should be given an inclusive rather than exclusive interpretation. For example, unless specifically noted, the term “a” should not be understood to mean “exactly one” or “one and only one”; instead, the term “a” means “one or more” or “at least one,” whether used in the claims or elsewhere in the specification and regardless of uses of quantifiers such as “at least one,” “one or more,” or “a plurality” elsewhere in the claims or specification.

The term “comprising” as used herein should be given an inclusive rather than exclusive interpretation. For example, a computer comprising one or more processors should not be interpreted as excluding other computer components, and may possibly include such components as memory, input/output devices, and/or network interfaces, among others.

While the above detailed description has shown, described, and pointed out novel features as applied to various implementations, it may be understood that various omissions, substitutions, and changes in the form and details of the devices or processes illustrated may be made without departing from the spirit of the disclosure. As may be recognized, certain implementations of the inventions described herein may be embodied within a form that does not provide all of the features and benefits set forth herein, as some features may be used or practiced separately from others. Each of the disclosed aspects and examples of the present disclosure may be considered individually or in combination with other aspects, examples, and variations of the disclosure. The headings used herein are merely provided to enhance readability and are not intended to limit the scope of the embodiments disclosed in a particular section to the features or elements disclosed in that section. The features or elements from one embodiment of the disclosure can be employed by other embodiments of the disclosure. For example, features described in one figure may be used in conjunction with embodiments illustrated in other figures. The foregoing description and examples have been set forth merely to illustrate the disclosure and are not intended as being limiting. The scope of certain inventions disclosed herein is indicated by the appended claims rather than by the foregoing description. All changes which come within the meaning and range of equivalency of the claims are to be embraced within their scope.

Some example embodiments are included below for illustrative purposes. These examples should not be viewed as limiting.

In a 1st Example, a hemodynamic sensor system is configured to determine a likelihood of effectiveness of an intervention for hypotension, the system comprising: a hemodynamic sensor that produces analog hemodynamic sensor signals representative of arterial pressure signal waveforms of one or more patients; an analog-to-digital converter that converts the analog hemodynamic sensor signals to arterial pressure signal waveforms; a graphical user interface configured to display an alert indicating the determined likelihood of effectiveness of the intervention; a non-transitory memory having executable instructions and a deep learning model stored thereon; and an electronic hardware processor in communication with the non-transitory memory and configured to execute the instructions to cause the system to at least: receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters, wherein the set of available heart health parameters comprises: a mean arterial pressure (MAP); a stroke volume index (SVI); a hypotension prediction index (HPI); a systemic vascular resistance (SVR); a heart rate (HR); a cardiac output (CO); a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV); obtain a plurality of reference arterial pressure signal waveforms from a plurality of patients; extract from the plurality of reference arterial pressure signal waveforms a plurality of reference sets of heart health parameters, each of the reference sets of heart health parameters comprising corresponding heart health parameters from the set of available heart health parameters; apply a filter to the reference sets of heart health parameters to generate a feature map associated with the filter; consolidate the filtered sets of reference heart health parameters into corresponding reference feature pools; select a reference maximum feature from each of the reference feature pools; reduce overfitting of the reference feature pools by removing one or more of the reference feature pools from remaining reference feature pools; normalize the selected reference maximum features from the reference feature pools; generate, based on the plurality of heart health parameters, one or more feature pools; determine normalized test maximum features from the plurality of test heart health parameters; determine, based on a comparison between the normalized selected reference maximum features and the normalized test maximum features, that an intervention for hypotension has been implemented and the likelihood of effectiveness of the intervention for hypotension of the patient; and generate, based on the determined likelihood of effectiveness of the intervention for hypotension of the patient, data for displaying, via the graphical user interface, the alert indicating the likelihood of effectiveness of the intervention for hypotension.

In a 2nd Example, a hemodynamic sensor system configured to determine a likelihood of effectiveness of an intervention for hypotension, the system comprising: a hemodynamic sensor that produces analog hemodynamic sensor signals representative of arterial pressure signal waveforms of one or more patients; an analog-to-digital converter that converts the analog hemodynamic sensor signals to arterial pressure signal waveforms; a non-transitory memory having executable instructions and a deep learning model stored thereon; and an electronic hardware processor in communication with the non-transitory memory and configured to execute the instructions to cause the system to at least: receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters; generate, using a plurality of reference arterial pressure signal waveforms from a plurality of patients, one or more filtered sets of reference heart health parameters associated with the plurality of reference arterial pressure signal waveforms; consolidate the filtered sets of reference heart health parameters into corresponding reference feature pools; determine, based on the reference feature pools, normalized test maximum features from the plurality of test heart health parameters; determine, based on the normalized test maximum features, that an intervention for hypotension has been implemented and the likelihood of effectiveness of the intervention for hypotension of the patient; and generate, based on the determined likelihood of effectiveness of the intervention for hypotension of the patient, data for displaying an alert indicating the likelihood of effectiveness of the intervention for hypotension.

In a 3rd Example, the hemodynamic sensor system of Example 2, wherein generating the one or more filtered sets of reference heart health parameters comprises: obtaining the plurality of reference arterial pressure signal waveforms from the plurality of patients; extracting from the plurality of reference arterial pressure signal waveforms a plurality of reference sets of heart health parameters, each of the reference sets of heart health parameters comprising corresponding heart health parameters from the set of available heart health parameters; and applying a filter to the reference sets of heart health parameters to generate a feature map associated with the filter.

In a 4th Example, the hemodynamic sensor system of any of Examples 2-3, wherein determining the normalized test maximum features from the plurality of test heart health parameters comprises: selecting a reference maximum feature from each of the reference feature pools; reducing overfitting of the reference feature pools by removing one or more of the reference feature pools from remaining reference feature pools; normalizing the selected reference maximum features from the reference feature pools; and generating, based on the plurality of heart health parameters, one or more feature pools.

In a 5th Example, the hemodynamic sensor system of any of Examples 2-4, further comprising a graphical user interface configured to display the alert indicating the determined likelihood of effectiveness of the intervention.

In a 6th Example, the hemodynamic sensor system of Example 5, wherein the electronic hardware processor is further configured to execute the instructions to cause the system to at least: generate, based on the determined likelihood of effectiveness of the intervention, data for displaying at least the alert indicating the likelihood of effectiveness of the intervention for hypotension.

In a 7th Example, the hemodynamic sensor system of any of Examples 2-6, wherein the alert comprises at least one of a symbol, a numerical value, a visual design, a repeated indicator, or a highlight.

In an 8th Example, the hemodynamic sensor system of any of Examples 2-7, wherein the electronic hardware processor is further configured to execute the instructions to cause the system to at least: determine, based on the normalized test maximum features, that the patient will experience hypotension within a specified time with at least a target threshold confidence level.

In a 9th Example, the hemodynamic sensor system of Example 8, wherein the electronic hardware processor is further configured to execute the instructions to cause the system to at least: generate, based on the determination that that the patient will experience hypotension within the specified time with at least the target threshold confidence level, data for displaying, via a graphical user interface, the alert indicating an expected time to event of hypotension within the patient.

In a 10th Example, the hemodynamic sensor system of any of Examples 8-9, further comprising an infusion pump, wherein the electronic hardware processor is further configured to execute the instructions to cause the system to at least: generate a control signal for the infusion pump to deliver an intravenous therapy to the patient based on the likelihood of effectiveness of the intervention for hypotension of the patient.

In a 11th Example, a hemodynamic sensor system configured to determine a likelihood that a patient will experience hypotension within a specified time with at least a target threshold confidence level, the system comprising: a hemodynamic sensor that produces analog hemodynamic sensor signals representative of arterial pressure signal waveforms of patients; an analog-to-digital converter that converts the analog hemodynamic sensor signals to arterial pressure signal waveforms; a non-transitory memory having executable instructions and a deep learning model stored thereon; and an electronic hardware processor in communication with the non-transitory memory and configured to execute the instructions to cause the system to at least: receive the specified time and receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters; generate, using a plurality of reference arterial pressure signal waveforms from a plurality of patients, one or more filtered sets of reference heart health parameters associated with the plurality of reference arterial pressure signal waveforms; consolidate the filtered sets of reference heart health parameters into corresponding reference feature pools; determine, based on the reference feature pools, normalized test maximum features from the plurality of test heart health parameters; determine, based on the normalized test maximum features, the likelihood that the patient will experience hypotension within the specified time with at least the target threshold confidence level; and generate, based on the determination of the likelihood that that the patient will experience hypotension within the specified time with at least the target threshold confidence level, data for displaying an alert indicating an expected time to event of hypotension within the patient.

In a 12th Example, the hemodynamic sensor system of Example 11, wherein generating the one or more filtered sets of reference heart health parameters comprises: obtaining the plurality of reference arterial pressure signal waveforms from the plurality of patients; extracting from the plurality of reference arterial pressure signal waveforms a plurality of reference sets of heart health parameters, each of the reference sets of heart health parameters comprising corresponding heart health parameters from the set of available heart health parameters; and applying a filter to the reference sets of heart health parameters to generate a feature map associated with the filter.

In a 13th Example, the hemodynamic sensor system of any of Examples 11-12, wherein determining the normalized test maximum features from the plurality of test heart health parameters comprises: selecting a reference maximum feature from each of the reference feature pools; reducing overfitting of the reference feature pools by removing one or more of the reference feature pools from remaining reference feature pools; normalizing the selected reference maximum features from the reference feature pools; and generating, based on the plurality of heart health parameters, one or more feature pools.

In a 14th Example, the hemodynamic sensor system of any of Examples 11-13, further comprising a graphical user interface configured to display the alert indicating an expected time to event of hypotension within the patient.

In a 15th Example, the hemodynamic sensor system of any of Examples 11-14, further comprising an infusion pump configured to deliver an intravenous therapeutic agent to the patient based on the likelihood that that the patient will experience hypotension within the specified time with at least the target threshold confidence level.

In a 16th Example, a hemodynamic sensor system configured to determine a likelihood that a patient will experience hypotension within a specified time with at least a target threshold confidence level, the system comprising: a hemodynamic sensor that produces analog hemodynamic sensor signals representative of arterial pressure signal waveforms of one or more patients; an analog-to-digital converter that converts the analog hemodynamic sensor signals to arterial pressure signal waveforms; a graphical user interface configured to display an alert indicating an expected time to event of hypotension within the patient; a non-transitory memory having executable instructions and a deep learning model stored thereon; and an electronic hardware processor in communication with the non-transitory memory and configured to execute the instructions to cause the system to at least: receive the specified time and receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters; obtain a plurality of reference arterial pressure signal waveforms from a plurality of patients; extract from the plurality of reference arterial pressure signal waveforms a plurality of reference sets of heart health parameters, each of the reference sets of heart health parameters comprising corresponding heart health parameters from the set of available heart health parameters; apply a filter to the reference sets of heart health parameters to generate a feature map associated with the filter; consolidate the filtered sets of reference heart health parameters into corresponding reference feature pools; select a reference maximum feature from each of the reference feature pools; reduce overfitting of the reference feature pools by removing one or more of the reference feature pools from remaining reference feature pools; normalize the selected reference maximum features from the reference feature pools; generate, based on the plurality of heart health parameters, one or more feature pools; determine normalized test maximum features from the plurality of test heart health parameters; determine, based on a comparison between the normalized selected reference maximum features and the normalized test maximum features, the likelihood that the patient will experience hypotension within the specified time with at least the target threshold confidence level; and generate, based on the determination of the likelihood that that the patient will experience hypotension within the specified time with at least the target threshold confidence level, data for displaying, via the graphical user interface, the alert indicating the expected time to event of hypotension within the patient.

In a 17th Example, a hemodynamic sensor system configured to determine a likelihood that a patient will experience hypotension within a specified time with at least a target threshold confidence level and determine a likelihood of effectiveness of an intervention for the hypotension, the system comprising: a hemodynamic sensor that produces analog hemodynamic sensor signals representative of arterial pressure signal waveforms of one or more patients; an analog-to-digital converter that converts the analog hemodynamic sensor signals to arterial pressure signal waveforms; a non-transitory memory having executable instructions and a deep learning model stored thereon; and an electronic hardware processor in communication with the non-transitory memory and configured to execute the instructions to cause the system to at least: receive the specified time; receive, from the hemodynamic sensor, a test analog hemodynamic sensor signal from the patient; convert, using the analog-to-digital converter, the test analog hemodynamic sensor signal to a test arterial pressure signal waveform; extract from the test arterial pressure signal waveform a plurality of test heart health parameters from a set of available heart health parameters; generate, using a plurality of reference arterial pressure signal waveforms from a plurality of patients, one or more filtered sets of reference heart health parameters associated with the plurality of reference arterial pressure signal waveforms; consolidate the filtered sets of reference heart health parameters into corresponding reference feature pools; determine, based on the reference feature pools, normalized test maximum features from the plurality of test heart health parameters; determine, based on the normalized test maximum features, the likelihood that the patient will experience hypotension within the specified time with at least the target threshold confidence level; determine, based on the normalized test maximum features, that an intervention for hypotension has been implemented and the likelihood of effectiveness of the intervention for hypotension of the patient; determine a hypotension prediction index (HPI) based on the determination of the likelihood that the patient will experience hypotension within the specified time with at least the target threshold confidence level and based on the likelihood of effectiveness of the intervention for hypotension of the patient; determine that the HPI exceeds a predetermined threshold; and generate, based on the determination that the HPI exceeds the predetermined threshold, an alert for display via a graphical user interface.

In an 18th Example, the hemodynamic sensor system of Example 17, further comprising the graphical user interface configured to display the alert indicating at least one of an expected time to event of hypotension within the patient or an indication of effectiveness of an intervention.

In a 19th Example, the hemodynamic sensor system of Example 18, wherein the alert is displayed via the graphical user interface.

In a 20th Example, the hemodynamic sensor system of any of Examples 17-19, wherein the set of available heart health parameters comprises: a mean arterial pressure (MAP); a stroke volume index (SVI); a hypotension prediction index (HPI); a systemic vascular resistance (SVR); a heart rate (HR); a cardiac output (CO); a time-based change in arterial pressure; a cardiac index (CI); a systemic vascular resistance index (SVRI); a normalized area of pulse pressure; an average distance between subsequent MAPs; an average distance between a systolic peak and a respective diastolic peak; and a stroke volume variation (SVV).

In a 21^{st} Example, the hemodynamic sensor system of Examples 17-20, wherein generating the one or more filtered sets of reference heart health parameters comprises: obtaining the plurality of reference arterial pressure signal waveforms from the plurality of patients; extracting from the plurality of reference arterial pressure signal waveforms a plurality of reference sets of heart health parameters, each of the reference sets of heart health parameters comprising corresponding heart health parameters from the set of available heart health parameters; and applying a filter to the reference sets of heart health parameters to generate a feature map associated with the filter.

In a 22nd Example, the hemodynamic sensor system of Examples 17-21, wherein determining the normalized test maximum features from the plurality of test heart health parameters comprises: selecting a reference maximum feature from each of the reference feature pools; reducing overfitting of the reference feature pools by removing one or more of the reference feature pools from remaining reference feature pools; normalizing the selected reference maximum features from the reference feature pools; and generating, based on the plurality of heart health parameters, one or more feature pools.

In a 23rd Example, the hemodynamic sensor system of Examples 17-22, further comprising an infusion pump configured to deliver an intravenous therapeutic agent to the patient based on the likelihood that that the patient will experience hypotension within the specified time with at least the target threshold confidence level.

What is claimed is:

1. A hemodynamic sensor system configured to determine a likelihood of effectiveness of an intervention for hypotension, the system comprising:
a hemodynamic sensor that produces analog hemodynamic sensor signals representative of arterial pressure signal waveforms of one or more patients;
an analog-to-digital converter that converts the analog hemodynamic sensor signals to arterial pressure signal waveforms;
a graphical user interface configured to display an alert indicating the determined likelihood of effectiveness of the intervention;
a non-transitory memory having executable instructions and a deep learning model stored thereon; and
an electronic hardware processor in communication with the non-transitory memory and configured to execute the instructions to cause the system to at least:
receive, from the hemodynamic sensor, a test analog hemodynamic sensor signal from the patient;
convert, using the analog-to-digital converter, the test analog hemodynamic sensor signal to a test arterial pressure signal waveform;
extract from the test arterial pressure signal waveform a plurality of test heart health parameters from a set of available heart health parameters, wherein the set of available heart health parameters comprises:
a mean arterial pressure (MAP);
a stroke volume index (SVI);
a hypotension prediction index (HPI);
a systemic vascular resistance (SVR);
a heart rate (HR);
a cardiac output (CO);
a time-based change in arterial pressure;
a cardiac index (CI);
a systemic vascular resistance index (SVRI);
a normalized area of pulse pressure;
an average distance between subsequent MAPs;
an average distance between a systolic peak and a respective diastolic peak; and
a stroke volume variation (SVV);
obtain a plurality of reference arterial pressure signal waveforms from a plurality of patients;
extract from the plurality of reference arterial pressure signal waveforms a plurality of reference sets of heart health parameters, each of the reference sets of heart health parameters comprising corresponding heart health parameters from the set of available heart health parameters;
apply a filter to the reference sets of heart health parameters to generate a feature map associated with the filter;
consolidate the filtered sets of reference heart health parameters into corresponding reference feature pools;
select a reference maximum feature from each of the reference feature pools;
reduce overfitting of the reference feature pools by removing one or more of the reference feature pools from remaining reference feature pools;
normalize the selected reference maximum features from the reference feature pools;
generate, based on the plurality of heart health parameters, one or more feature pools;
determine normalized test maximum features from the plurality of test heart health parameters;
determine, based on a comparison between the normalized selected reference maximum features and the normalized test maximum features, that an intervention for hypotension has been implemented and the likelihood of effectiveness of the intervention for hypotension of the patient; and
generate, based on the determined likelihood of effectiveness of the intervention for hypotension of the patient, data for displaying, via the graphical user interface, the alert indicating the likelihood of effectiveness of the intervention for hypotension.

2. A hemodynamic sensor system configured to determine a likelihood of effectiveness of an intervention for hypotension, the system comprising:
a hemodynamic sensor that produces analog hemodynamic sensor signals representative of arterial pressure signal waveforms of one or more patients;
an analog-to-digital converter that converts the analog hemodynamic sensor signals to arterial pressure signal waveforms;
a non-transitory memory having executable instructions and a deep learning model stored thereon; and
an electronic hardware processor in communication with the non-transitory memory and configured to execute the instructions to cause the system to at least:
receive, from the hemodynamic sensor, a test analog hemodynamic sensor signal from the patient;
convert, using the analog-to-digital converter, the test analog hemodynamic sensor signal to a test arterial pressure signal waveform;
extract from the test arterial pressure signal waveform a plurality of test heart health parameters from a set of available heart health parameters;
generate, using a plurality of reference arterial pressure signal waveforms from a plurality of patients, one or more filtered sets of reference heart health parameters associated with the plurality of reference arterial pressure signal waveforms;
consolidate the filtered sets of reference heart health parameters into corresponding reference feature pools;
determine, based on the reference feature pools, normalized test maximum features from the plurality of test heart health parameters;
determine, based on the normalized test maximum features, that an intervention for hypotension has been implemented and the likelihood of effectiveness of the intervention for hypotension of the patient; and
generate, based on the determined likelihood of effectiveness of the intervention for hypotension of the patient, data for displaying an alert indicating the likelihood of effectiveness of the intervention for hypotension.

3. The hemodynamic sensor system of claim 2, wherein generating the one or more filtered sets of reference heart health parameters comprises:
obtaining the plurality of reference arterial pressure signal waveforms from the plurality of patients;
extracting from the plurality of reference arterial pressure signal waveforms a plurality of reference sets of heart health parameters, each of the reference sets of heart health parameters comprising corresponding heart health parameters from the set of available heart health parameters; and
applying a filter to the reference sets of heart health parameters to generate a feature map associated with the filter.

4. The hemodynamic sensor system of claim 2, wherein determining the normalized test maximum features from the plurality of test heart health parameters comprises:
selecting a reference maximum feature from each of the reference feature pools;
reducing overfitting of the reference feature pools by removing one or more of the reference feature pools from remaining reference feature pools;
normalizing the selected reference maximum features from the reference feature pools; and
generating, based on the plurality of heart health parameters, one or more feature pools.

5. The hemodynamic sensor system of claim 2, further comprising a graphical user interface configured to display the alert indicating the determined likelihood of effectiveness of the intervention.

6. The hemodynamic sensor system of claim 5, wherein the electronic hardware processor is further configured to execute the instructions to cause the system to at least:
generate, based on the determined likelihood of effectiveness of the intervention, data for displaying at least the alert indicating the likelihood of effectiveness of the intervention for hypotension.

7. The hemodynamic sensor system of claim 2, wherein the alert comprises at least one of a symbol, a numerical value, a visual design, a repeated indicator, or a highlight.

8. The hemodynamic sensor system of claim 2, wherein the electronic hardware processor is further configured to execute the instructions to cause the system to at least:
determine, based on the normalized test maximum features, that the patient will experience hypotension within a specified time with at least a target threshold confidence level.

9. The hemodynamic sensor system of claim 8, wherein the electronic hardware processor is further configured to execute the instructions to cause the system to at least:
generate, based on the determination that that the patient will experience hypotension within the specified time with at least the target threshold confidence level, data for displaying, via a graphical user interface, the alert indicating an expected time to event of hypotension within the patient.

10. The hemodynamic sensor system of claim 8, further comprising an infusion pump, wherein the electronic hardware processor is further configured to execute the instructions to cause the system to at least:
generate a control signal for the infusion pump to deliver an intravenous therapy to the patient based on the likelihood of effectiveness of the intervention for hypotension of the patient.

11. A hemodynamic sensor system configured to determine a likelihood that a patient will experience hypotension within a specified time with at least a target threshold confidence level, the system comprising:
a hemodynamic sensor that produces analog hemodynamic sensor signals representative of arterial pressure signal waveforms of patients;
an analog-to-digital converter that converts the analog hemodynamic sensor signals to arterial pressure signal waveforms;
a non-transitory memory having executable instructions and a deep learning model stored thereon; and
an electronic hardware processor in communication with the non-transitory memory and configured to execute the instructions to cause the system to at least:
receive the specified time and
receive, from the hemodynamic sensor, a test analog hemodynamic sensor signal from the patient;
convert, using the analog-to-digital converter, the test analog hemodynamic sensor signal to a test arterial pressure signal waveform;
extract from the test arterial pressure signal waveform a plurality of test heart health parameters from a set of available heart health parameters;
generate, using a plurality of reference arterial pressure signal waveforms from a plurality of patients, one or more filtered sets of reference heart health parameters associated with the plurality of reference arterial pressure signal waveforms;
consolidate the filtered sets of reference heart health parameters into corresponding reference feature pools;
determine, based on the reference feature pools, normalized test maximum features from the plurality of test heart health parameters;
determine, based on the normalized test maximum features, the likelihood that the patient will experience hypotension within the specified time with at least the target threshold confidence level; and
generate, based on the determination of the likelihood that that the patient will experience hypotension within the specified time with at least the target threshold confidence level, data for displaying an alert indicating an expected time to event of hypotension within the patient.

12. The hemodynamic sensor system of claim 11, wherein generating the one or more filtered sets of reference heart health parameters comprises:
obtaining the plurality of reference arterial pressure signal waveforms from the plurality of patients;
extracting from the plurality of reference arterial pressure signal waveforms a plurality of reference sets of heart health parameters, each of the reference sets of heart health parameters comprising corresponding heart health parameters from the set of available heart health parameters; and
applying a filter to the reference sets of heart health parameters to generate a feature map associated with the filter.

13. The hemodynamic sensor system of claim 11, wherein determining the normalized test maximum features from the plurality of test heart health parameters comprises:
selecting a reference maximum feature from each of the reference feature pools;
reducing overfitting of the reference feature pools by removing one or more of the reference feature pools from remaining reference feature pools;
normalizing the selected reference maximum features from the reference feature pools; and
generating, based on the plurality of heart health parameters, one or more feature pools.

14. The hemodynamic sensor system of claim 11, further comprising a graphical user interface configured to display the alert indicating an expected time to event of hypotension within the patient.

15. The hemodynamic sensor system of claim 11, further comprising an infusion pump configured to deliver an intravenous therapeutic agent to the patient based on the likelihood that that the patient will experience hypotension within the specified time with at least the target threshold confidence level.

16. A hemodynamic sensor system configured to determine a likelihood that a patient will experience hypotension within a specified time with at least a target threshold confidence level, the system comprising:
a hemodynamic sensor that produces analog hemodynamic sensor signals representative of arterial pressure signal waveforms of one or more patients;
an analog-to-digital converter that converts the analog hemodynamic sensor signals to arterial pressure signal waveforms;
a graphical user interface configured to display an alert indicating an expected time to event of hypotension within the patient;
a non-transitory memory having executable instructions and a deep learning model stored thereon; and
an electronic hardware processor in communication with the non-transitory memory and configured to execute the instructions to cause the system to at least:
receive the specified time and
receive, from the hemodynamic sensor, a test analog hemodynamic sensor signal from the patient;
convert, using the analog-to-digital converter, the test analog hemodynamic sensor signal to a test arterial pressure signal waveform;
extract from the test arterial pressure signal waveform a plurality of test heart health parameters from a set of available heart health parameters;
obtain a plurality of reference arterial pressure signal waveforms from a plurality of patients;
extract from the plurality of reference arterial pressure signal waveforms a plurality of reference sets of heart health parameters, each of the reference sets of heart health parameters comprising corresponding heart health parameters from the set of available heart health parameters;
apply a filter to the reference sets of heart health parameters to generate a feature map associated with the filter;
consolidate the filtered sets of reference heart health parameters into corresponding reference feature pools;
select a reference maximum feature from each of the reference feature pools;
reduce overfitting of the reference feature pools by removing one or more of the reference feature pools from remaining reference feature pools;
normalize the selected reference maximum features from the reference feature pools;
generate, based on the plurality of heart health parameters, one or more feature pools;
determine normalized test maximum features from the plurality of test heart health parameters;
determine, based on a comparison between the normalized selected reference maximum features and the normalized test maximum features, the likelihood that the patient will experience hypotension within the specified time with at least the target threshold confidence level; and
generate, based on the determination of the likelihood that that the patient will experience hypotension within the specified time with at least the target threshold confidence level, data for displaying, via the graphical user interface, the alert indicating the expected time to event of hypotension within the patient.

17. A hemodynamic sensor system configured to determine a likelihood that a patient will experience hypotension within a specified time with at least a target threshold confidence level and determine a likelihood of effectiveness of an intervention for the hypotension, the system comprising:
a hemodynamic sensor that produces analog hemodynamic sensor signals representative of arterial pressure signal waveforms of one or more patients;
an analog-to-digital converter that converts the analog hemodynamic sensor signals to arterial pressure signal waveforms;
a non-transitory memory having executable instructions and a deep learning model stored thereon; and
an electronic hardware processor in communication with the non-transitory memory and configured to execute the instructions to cause the system to at least:
receive the specified time;
receive, from the hemodynamic sensor, a test analog hemodynamic sensor signal from the patient;
convert, using the analog-to-digital converter, the test analog hemodynamic sensor signal to a test arterial pressure signal waveform;
extract from the test arterial pressure signal waveform a plurality of test heart health parameters from a set of available heart health parameters;
generate, using a plurality of reference arterial pressure signal waveforms from a plurality of patients, one or more filtered sets of reference heart health parameters associated with the plurality of reference arterial pressure signal waveforms;
consolidate the filtered sets of reference heart health parameters into corresponding reference feature pools;
determine, based on the reference feature pools, normalized test maximum features from the plurality of test heart health parameters;
determine, based on the normalized test maximum features, the likelihood that the patient will experience hypotension within the specified time with at least the target threshold confidence level;
determine, based on the normalized test maximum features, that an intervention for hypotension has been implemented and the likelihood of effectiveness of the intervention for hypotension of the patient;
determine a hypotension prediction index (HPI) based on the determination of the likelihood that the patient will experience hypotension within the specified time with at least the target threshold confidence level and based on the likelihood of effectiveness of the intervention for hypotension of the patient;
determine that the HPI exceeds a predetermined threshold; and
generate, based on the determination that the HPI exceeds the predetermined threshold, an alert for display via a graphical user interface.

18. The hemodynamic sensor system of claim 17, further comprising the graphical user interface configured to display the alert indicating at least one of an expected time to event of hypotension within the patient or an indication of effectiveness of an intervention.

19. The hemodynamic sensor system of claim 18, wherein the alert is displayed via the graphical user interface.

20. The hemodynamic sensor system of claim 17, wherein the set of available heart health parameters comprises:
a mean arterial pressure (MAP);
a stroke volume index (SVI);
a hypotension prediction index (HPI);
a systemic vascular resistance (SVR);
a heart rate (HR);
a cardiac output (CO);
a time-based change in arterial pressure;
a cardiac index (CI);
a systemic vascular resistance index (SVRI);
a normalized area of pulse pressure;
an average distance between subsequent MAPs;
an average distance between a systolic peak and a respective diastolic peak; and
a stroke volume variation (SVV).

21. The hemodynamic sensor system of claim 17, wherein generating the one or more filtered sets of reference heart health parameters comprises:
obtaining the plurality of reference arterial pressure signal waveforms from the plurality of patients;
extracting from the plurality of reference arterial pressure signal waveforms a plurality of reference sets of heart health parameters, each of the reference sets of heart health parameters comprising corresponding heart health parameters from the set of available heart health parameters; and
applying a filter to the reference sets of heart health parameters to generate a feature map associated with the filter.

22. The hemodynamic sensor system of claim 17, wherein determining the normalized test maximum features from the plurality of test heart health parameters comprises:
selecting a reference maximum feature from each of the reference feature pools;
reducing overfitting of the reference feature pools by removing one or more of the reference feature pools from remaining reference feature pools;
normalizing the selected reference maximum features from the reference feature pools; and
generating, based on the plurality of heart health parameters, one or more feature pools.

23. The hemodynamic sensor system of claim 17, further comprising an infusion pump configured to deliver an intravenous therapeutic agent to the patient based on the likelihood that that the patient will experience hypotension within the specified time with at least the target threshold confidence level.
